(19) United States
(12) Patent Application Publication
(10) Pub. No.: US 2026/0248465 A1
(43) Pub. Date: Aug. 27, 2026

(54) AN INTERACTIVE USER INTERFACE, AND METHODS AND SYSTEMS FOR PROVIDING THE INTERACTIVE USER INTERFACE

(71) Applicant: Human CHIMP Pty Ltd, Victoria (AU)

(72) Inventors: Stephen Frederick WOODFORD, Victoria (AU); Mathew John DAVIS, Victoria (AU); Ruth MARSHALL, Victoria (AU)

(21) Appl. No.: 19/163,362

(22) PCT Filed: Mar. 8, 2024
(86) PCT No.: PCT/AU2024/050208

§ 371 Date: Sep. 8, 2025

(30) Foreign Application Priority Data
Mar. 10, 2023 (AU) 2023900656

(51) Int. Cl. A61B 5/00; A61B 5/02; A61B 5/021; A61B 5/024; G16H 50/30

(52) U.S. Cl. CPC A61B 5/7445; A61B 5/02028; A61B 5/7475; G16H 50/30; A61B 5/021; A61B 5/024

(57) ABSTRACT

Described embodiments comprise determining one or more data points of a time series, each of the one or more data points comprising values for each of a first, second and third variable. Each of the first, second and third variables are indicative of health information of a subject. The method further comprises providing an interactive user interface on a display screen of a device. The interactive user interface comprises a visual representation defining a 3D space, wherein a first edge of the visual representation is representative of the first variable, a second edge of the visual representation is representative of the second variable, and a third edge of the visual representation is representative of the third variable. The method further comprises plotting the one or more data points in the 3D space such that a first face of the visual representation defined by the first and second edges depicts a first health measure defined by a function of the first and second variables, a second face of the visual representation defined by the first and third edges depicts a second health measure defined by a function of the first and third variables, and a third face of the visual representation defined by the second and third edges depicts a third health measure defined by a function of the first and third variables. The interactive user interface is configured to facilitate orientation of the visual representation to allow different views of the visual representation and accordingly, the first, second and third health variables and/or measures to be presented, and responsive to receiving an orientation input from a user via the interactive user interface, the method comprises orientating the visual representation in accordance with the orientation input.

### TECHNICAL FIELD

Described embodiments relate to computer-implemented methods and computing systems for generating and providing an interactive user interface. In some embodiments, the methods and systems relate to generating interactive 3D user interfaces of haemodynamic performance data of a subject.

### BACKGROUND

Known methods and systems for generating and depicting health data tend to provide a number of individual and/or disparate user interfaces to build an understanding of the health status of a patient. A physician or other clinical expert is then tasked with interpreting the information as presented by the separate user interfaces to make an assessment of the health status of the patient. This may lead to inconsistent, incomplete and/or inaccurate determinations of a patient's health status, which may lead to sub-optimal outcomes for the patient.

It is desired to address or ameliorate some of the disadvantages associated with such prior methods and systems, or at least to provide a useful alternative thereto.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

Some embodiments are directed to a method comprising: determining one or more data points of a time series, each of the one or more data points comprising values for each of a first, second and third variable, each of the first, second and third variables being indicative of health information of a subject; providing an interactive user interface on a display screen of a device, the interactive user interface comprising a visual representation defining a 3D space, wherein a first edge of the visual representation is representative of the first variable, a second edge of the visual representation is representative of the second variable, and a third edge of the visual representation is representative of the third variable; and plotting the one or more data points in the 3D space such that a first face of the visual representation defined by the first and second edges depicts a first health measure defined by a function of the first and second variables, a second face of the visual representation defined by the first and third edges depicts a second health measure defined by a function of the first and third variables, and a third face of the visual representation defined by the second and third edges depicts a third health measure defined by a function of the first and third variables; wherein the interactive user interface is configured to facilitate orientation of the visual representation to allow different views of the visual representation and accordingly, the first, second and third health variables and/or measures to be presented, and responsive to receiving an orientation input from a user via the interactive user interface, orientating the visual representation in accordance with the orientation input.

In some embodiments, plotting the one or more data points comprises plotting the one or more data points in time sequence order.

In some embodiments the one or more data points are measured at successive equally spaced points in time.

In some embodiments the one or more data points are plotted based on heart rate and/or cardiac cycles.

In some embodiments, collectively, the first, second and third variables and/or health measures depicted by the visual representation represent perfusion, for example, haemodynamic function, in the body of the subject.

In some embodiments, the first variable is indicative of stroke volume (SV), the second variable is indicative of heart rate (HR), and the first health measure is indicative of cardiac output (CO).

In some embodiments, the first variable is indicative of stroke volume (SV), the third variable is indicative of systemic elastance (Esys), and the second health measure is indicative of systemic perfusion pressure (SPP).

In some embodiments, the second variable is indicative of heart rate (HR), the third variable is indicative of systemic elastance (Esys), and the third health measure is systemic vascular resistance (SVR).

In some embodiments, the first variable is indicative of stroke volume (SV), the third variable is indicative of effective arterial elastance (Ea), and the second health measure is indicative of mean arterial pressure (MAP).

In some embodiments, the second variable is indicative of heart rate (HR), the third variable is indicative of effective arterial elastance (Ea), and the third health measure is total peripheral resistance (TPR).

In some embodiments, the first variable is indicative of systemic perfusion pressure (SPP), the second variable is indicative of indexed cardiac output (CI) and the first health measure is indicative of indexed cardiac power (CPI).

In some embodiments, the second variable is indicative of indexed cardiac output (CI), the third variable is indicative of heart rate (HR) and the third health measure is indicative of indexed stroke volume (SVI).

In some embodiments, the first variable is indicative of systemic perfusion pressure (SPP), the second variable is indicative of cardiac output (CO) and the first health measure is indicative of cardiac power (CP).

In some embodiments, the second variable is indicative of cardiac output (CO), the third variable is indicative of heart rate (HR), and the second health measure is indicative of stroke volume (SV).

In some embodiments, the first variable is indicative of systemic perfusion pressure (SPP), the third variable is indicative of heart rate (HR), and the third health measure is indicative of rate pressure product (RPP).

In some embodiments, the first variable is indicative of systemic perfusion pressure (SPP), the second variable is indicative of indexed stroke volume (SVI), and the first health measure is indicative of indexed left ventricular stroke work (LVSWI).

In some embodiments, the second variable is indicative of indexed stroke volume (SVI), the third variable is indicative of heart rate (HR), and the second health measure is indicative of indexed cardiac output (CI).

In some embodiments, the first variable is indicative of systemic perfusion pressure (SPP), the second variable is indicative of stroke volume (SV), and the first health measure is indicative of left ventricular stroke work (LVSW).

In some embodiments, the second variable is indicative of stroke volume (SV), the third variable is indicative of heart rate (HR), and the second health measure is indicative of cardiac output (CO).

In some embodiments, the first variable is indicative of right ventricle stroke volume (RVSV) as the first variable, the second variable is indicative of heart rate (HR), and the first health measure is indicative of right ventricle cardiac output (RVCO).

In some embodiments, the first variable is indicative of right ventricle stroke volume (RVSV), the third variable is indicative of pulmonary elastance (Ep) and the second health measure is indicative of pulmonary perfusion pressure (PPP).

In some embodiments, the second variable is indicative of heart rate (HR), the third variable is indicative of pulmonary elastance (Ep) and the third health measure is indicative of pulmonary vascular resistance (PVR).

In some embodiments, the first variable is indicative of pulmonary perfusion pressure (PPP), the second variable is indicative of right ventricle stroke volume (RVSV), and the first health measure is indicative of right ventricle stroke work (RVSW) or pulmonary elastance (Ep).

In some embodiments, the first variable is pulmonary perfusion pressure (PPP), the third variable is indicative of heart rate (HR) and the second health measure is indicative of right ventricle rate pressure product (RVRPP).

In some embodiments, the second variable is indicative of right ventricle stroke volume (RVSV), the third variable is indicative of heart rate (HR) and the third health measure is indicative of right ventricle cardiac output (RVCO).

In some embodiments, the first variable is indicative of pulmonary perfusion pressure (PPP) as the first variable, the second variable is indicative of right ventricle stroke volume index (RVSVI), and the first health measure is indicative of right ventricle stroke work index (RVSWI).

In some embodiments, the first variable is pulmonary perfusion pressure (PPP), the third variable is indicative of heart rate (HR) and the second health measure is indicative of right ventricle rate pressure product (RVRPP).

In some embodiments, the second variable is indicative of right ventricle stroke volume index (RVSVI), the third variable is indicative of heart rate (HR) and the third health measure is indicative of right ventricle cardiac index (RVCI).

In some embodiments, the first variable is indicative of pulmonary perfusion pressure (PPP), the second variable is indicative of right ventricle cardiac output (RVCO), and the first health measure is indicative of pulmonary vascular resistance (PVR) or right ventricular power (RVP).

In some embodiments, the first variable is pulmonary perfusion pressure (PPP), the third variable is indicative of heart rate (HR) and the second health measure is indicative of right ventricle rate pressure product (RVRPP).

In some embodiments, the second variable is indicative of right ventricle cardiac output (RVCO), the third variable is indicative of heart rate (HR) and the third health measure is indicative of the right ventricle stroke volume (RVSV).

In some embodiments, the first variable is indicative of pulmonary perfusion pressure (PPP), the second variable is indicative of right ventricle cardiac index (RVCI), and the first health measure is indicative of pulmonary vascular resistance index (PVRI).

In some embodiments, the first variable is pulmonary perfusion pressure (PPP), the third variable is indicative of heart rate (HR) and the second health measure is indicative of right ventricle rate pressure product (RVRPP).

In some embodiments, the second variable is indicative of right ventricle cardiac index (RVCI), the third variable is indicative of heart rate (HR) and the third health measure is indicative of the right ventricle stroke volume index (RVSVI).

In some embodiments, the orientation input is a selection of one of the first, second or third face of the visual representation, and the method further comprises responsive to receiving the selection, causing the visual representation to depict, on one of the first, second or third face of the visual representation, a two-dimensional graph; plotting on the two-dimensional graph the data points, wherein the data points are indicative of the variables associated with the first, second or third face of the first visual representation; and wherein the two-dimensional graph is indicative of one of the first, second or third health measures associated with the first, second or third face of the first visual representation respectively.

In some embodiments, the two-dimensional graph further comprises one or more reference lines.

In some embodiments, the method comprises plotting at least a first reference line on the two-dimensional graph, the at least a first reference line configured to: (i) aid the user to determine value(s) of the data points plotted on the two-dimensional graph; or (ii) represent one or more health measures relative to the data points plotted on the two-dimensional graph.

In some embodiments, the method comprises plotting at least a first reference line on each of the first, second and/or third faces of the first visual representation, wherein the at least first reference lines of the first, second and/or third faces are indicative of different health measures, and wherein the at least first reference line is configured to (i) aid the user to determine value(s) of the data points plotted on the two-dimensional graph; or (ii) represent one or more health measures relative to the data points plotted on the respective first, second and/or third face. For example, when the at least first reference line is configured to represent one or more health measures, the one or more health measures comprising one or more of: SPP, CO, SVR, MAP, TPR, CI, SVRI, TPRI, PPP, RVCO, RVCI, PVR, or PVRI.

In some embodiments, the method comprises plotting a second reference line on each of the first, second and/or third face of the visual representation, wherein the second reference lines of the first, second and/or third faces are indicative of different health measures, and wherein the second reference line is configured to (i) aid the user to determine value(s) of the one or more data points plotted on the two-dimensional graph; or (ii) represent one or more health measures relative to the one or more data points plotted on the respective first, second and/or third face. For example, when the second reference line is configured to represent one or more health measures, one or more health measures may comprise one or more of: indexed systemic vascular resistance (SVRI); systemic vascular resistance (SVR); indexed systemic elastance (EsysI); systemic elastance (Esys); pulmonary elastance (Ep); indexed pulmonary elastance (EpI); right ventricle power (RVP); and right ventricle power index (RVPI).

In some embodiments, the data points are determined from biometric data that is indicative of one or more of variables or measures: systemic vascular resistance (SVR); heart rate (HR); central venous pressure (CVP); mean arterial pressure (MAP); stroke volume (SV); systemic perfusion pressure (SPP); systemic elastance (Esys); cardiac output (CO); total peripheral resistance (TPR); effective arterial elastance (Ea); cardiac cycle time (CC); mean pulmonary arterial pressure (MPAP); pulmonary capillary wedge pressure (PCWP); left atrial pressure (LAP); pulmonary perfusion pressure (PPP); pulmonary vascular resistance (PVR); pulmonary elastance (Ep); right ventricle stroke volume (RVSV); right ventricle cardiac output (RVCO); indexed stroke volume (SVI); indexed systemic vascular resistance (SVRI); indexed cardiac output (CI); indexed systemic elastance (EsysI); indexed effective arterial elastance (EaI); indexed total peripheral resistance (TPRI); indexed right ventricle stroke volume (RVSVI); indexed right ventricle cardiac output (RVCI); indexed pulmonary vascular resistance (PVRI); and indexed pulmonary elastance (EpI); wherein the one or more indexed variables is indexed based on a body surface area (BSA) of the subject. SV refers to the left ventricle stroke volume, and is at times estimated by the right ventricle stroke volume (RVSV).

In some embodiments, the 3D space further comprises a safe zone, being indicative of a range of safe values of the first, second and/or third variables, the safe zone comprising an outer boundary and being indicative of a region of the 3D space associated with a low possibility of injury to the subject.

In some embodiments, the method of any of the present disclosures further comprises: determining the positions of the one or more data points in the 3D space relative to the safe zone; determining, based on the positions of the one or more data points proximal to and/or outside of the outer boundary of the safe zone, an amount of time the first, second and/or third variables have been outside the range of safe values; wherein the amount of time is indicative of a risk of injury to the subject.

In some embodiments, the method of any of the present disclosures further comprises: determining, based on the amount of time the first, second and/or third variables have been outside the range of safe values a level of risk that the subject will be injured.

In some embodiments, the method of any of the present disclosures further comprises: presenting a notification on the display screen of the device indicative of the level of risk that the subject will be injured.

In some embodiments, plotting the one or more data points in the 3D space comprises plotting at least a plurality of data points and wherein the method of any of the present disclosures further comprises: determining that one or more of the variables of a set of the plurality of data points deviates from an expected zone for the respective variables; and responsive to determining that the one or more of the variables of the set of the plurality of data points deviates from the expected zone, determining that a system defect has occurred.

In some embodiments, determining that one or more of the variables of a set of the plurality of data points deviates from an expected zone for the respective variables comprises: determining that stroke volume has fallen below a first threshold, that systemic elastance has exceeded a second threshold, and that heart rate is relatively stable; and responsive to determining that stroke volume has fallen below a first threshold, that systemic elastance has exceeded a second threshold, and that heart rate is relatively stable, determining that an arterial line has become damped.

In some embodiments, responsive to determining that a system defect has occurred, presenting a notification on the display screen of the device indicative of the occurrence of the system defect.

In some embodiments, CC may be substituted for HR using the equation:

$$CC = \frac{1}{H R} \times 60$$

Some embodiments are directed to a system for providing an interactive user interface, the system comprising: a computing device configured to determine one or more data points of a time series, each of the one or more data points comprising values for each of a first, second and third variable, each of the first, second and third variables being indicative of health information of a subject; a display screen of a device, configured to display the interactive user interface, the interactive user interface comprising: a visual representation defining a 3D space, wherein a first edge of the visual representation is representative of the first variable, a second edge of the visual representation is representative of the second variable, and a third edge of the visual representation is representative of the third variable, wherein the one or more data points are plotted in the 3D space such that a first face of the visual representation defined by the first and second edges depicts a first health measure defined by a function of the first and second variables, a second face of the visual representation defined by the first and third edges depicts a second health measure defined by a function of the first and third variables, and a third face of the visual representation defined by the second and third edges depicts a third health measure defined by a function of the first and third variables; and wherein the interactive user interface is configured to: facilitate orientation of the visual representation to allow different views of the visual representation and accordingly, the first, second and third health measures to be presented; and in response to receiving an orientation input from a user via the device, orient the visual representation in accordance with the orientation input.

Some embodiments are directed to a non-transient computer-readable storage medium comprising executable code, which when executed by one or more processors, are configured to perform any one of the described methods.

Some embodiments are directed to a system comprising: one or more processors; and memory, comprising instructions, which when executed by the one or more processors, are configured to perform any one of the described methods.

Some embodiments are directed to a graphical user interface for display on a display screen of a device wherein the graphical user interface is configured for display in a window occupying all or a portion of the display screen and comprising: a visual representation defining a 3D space, wherein a first edge of the visual representation is representative of a first health variable, a second edge of the visual representation is representative of a second health variable, and a third edge of the visual representation is representative of a third health variable; wherein a first face of the visual representation defined by the first and second edges depicts a first health measure defined by a function of the first and second variables, a second face of the visual representation defined by the first and third edges depicts a second health measure defined by a function of the first and third variables, and a third face of the visual representation defined by the second and third edges depicts a third health measure defined by a function of the first and third variables; and wherein the 3D space comprises a plot of one or more data points of a time series, each of the one or more data points comprising values for each of a first, second and third health variable, each of the first, second and third variables being indicative of health information of a subject, such that a first face of the visual representation defined by the first and second edges depicts a first health measure defined by a function of the first and second variables, a second face of the visual representation defined by the first and third edges depicts a second health measure defined by a function of the first and third variables, and a third face of the visual representation defined by the second and third edges depicts a third health measure defined by a function of the first and third variables; and responsive to receiving an orientation input from a user via the interactive user interface, the graphical user interface orientates the visual representation in accordance with the orientation input.

### BRIEF DESCRIPTION OF DRAWINGS

Some embodiments will now be described by way of non-limiting examples with reference to the accompanying drawings.

FIG. 1 is a process flow diagram of a high-level overview of a method of generating and providing an interactive user interface, according to some embodiments;

FIG. 2 is a block diagram of a system for generating and providing an interactive user interface, according to some embodiments;

FIGS. 3A to 3E are block diagrams of the system for generating and providing an interactive user interface of FIG. 2, according to some embodiments;

FIG. 4 is a process flow diagram of a method for generating and providing an interactive user interface, according to some embodiments;

FIG. 5 is an example image of an interactive user interface, according to some embodiments;

FIG. 6 is an example image of a view of the interactive user interface of FIG. 5 showing a first health measure defined by two variables indicative of a health information of a subject, according to some embodiments;

FIG. 7 is an image of the interactive user interface of FIG. 5 showing a second health measure defined by two variables indicative of a health information of a subject, according to some embodiments;

FIG. 8 is an image of the interactive user interface of FIG. 5, showing a third health measure defined by two variables indicative of a health information of a subject, according to some embodiments;

FIG. 9 is an image of the interactive user interface of FIG. 5, showing a zone indicative of a health condition, according to some embodiments;

FIG. 10A is an image of the interactive user interface of FIG. 5, showing data points indicative of an abnormal system condition according to some embodiments;

FIG. 10B is an image of the interactive user interface of FIG. 7, showing data points indicative of an abnormal system condition according to some embodiments;

FIG. 10C is an image of the interactive user interface of FIG. 6, showing data points indicative of an abnormal system condition according to some embodiments; and

FIG. 10D is an image of the interactive user interface of FIG. 8, showing data points indicative of an abnormal system condition according to some embodiments.

### DESCRIPTION OF EMBODIMENTS

Described embodiments relate to computer-implemented methods and computing systems for generating and providing user interfaces, such as an interactive graphical user interface. In some embodiments, the methods and systems relate to user interfaces configured to represent perfusion measures of a subject, such as haemodynamic performance data.

The interactive graphical user interface comprises a visual representation, such as an object, block, or cube defining a 3D space. A first edge of the visual representation (e.g., cube) represents a first health variable, a second edge of the visual representation (e.g., cube) represents a second health variable, and a third edge of the visual representation (e.g., cube) represents a third health variable. A first face of the visual representation (e.g., cube), defined by the first and second edges depicts a first health measure, a second face of the visual representation (e.g., cube) defined by the first and third edges depicts a second health measure, and a third face of the visual representation (e.g., cube) defined by the second and third edges depicts a third health measure. In response to user input via the user interface, the visual representation (e.g., cube) can be orientated to present various views of the cube to the user.

In some embodiments, biometric data is determined from a subject, for example using monitoring devices. A plurality of data points of a time series is determined from the biometric data. The data points comprise values of health variables and/or measures. The data points are plotted in the 3D space defined by the visual representation (e.g., cube). The data points may be plotted in time sequence order such that the progression or change in value of the variables over time is readily discernible. In some embodiments, the plots may be scatterplots and/or line graphs.

This is much more preferable than generating and providing a plurality of individual or disparate user interfaces displaying haemodynamic performance data to allow a physician to gain an understanding of the health status of a patient. Although each individual user interface could present a health variable and/or measure defined by a single haemodynamic variable and/or measure over time, or in relation to other known haemodynamic variable and/or measure, due to the disparate nature of the information presented over the multiple interfaces, a physician or other clinical expert would be tasked with mentally combining the information as presented by the separate user interfaces to arrive at an overall determination of the health status of the patient. This would likely involve the physician or other clinical expert needing to perform a mental calibration or alignment of the disparate information to gain a meaningful view of the patient's health status, at one point in time, or over a period of time. This may lead to incomplete and/or inaccurate determinations of a patient's health status, which may lead to sub-optimal outcomes for the patient.

In contrast, the interactive user interface of the described embodiments are intuitive and allow for a more comprehensive interpretation of biometric data, the determination of a health status of a subject, and/or the making of clinical decisions. The collective representation of the three different health variables and three different health measures in a single cubic representation presents the user with more information than three disparate independent representations of the health variables and/or measures would in that their interrelationship is clearly depicted. Furthermore, due to the interactive nature of the user interface, a physician or clinician can interact with the cubic representation to orientate the views being shown allowing them to focus on any one of the faces of the visual representation (e.g., cube) representing the first, second and third health measures, or indeed any transitions between those faces, including views that show two of the three health measures or all three heath measures concurrently.

Perfusion is the delivery of oxygen from the lungs to the body's cells. Managing perfusion is a core concern of cardiovascular medicine. The blood, the heart, and the vasculature contribute to perfusion; and these three elements form part of both the ‘systemic circulation’ and the ‘pulmonary circulation’.

The systemic circulation despatches oxygen-rich blood from the left ventricle of the heart out through the systemic vasculature to the tissues and transports deoxygenated blood back to the heart. The pulmonary circulation works with the lungs to re-oxygenate blood: the right ventricle of the heart receives de-oxygenated blood from the systemic vasculature and pumps it through the pulmonary vasculature to the lungs, from where the re-oxygenated blood flows back to the left ventricle of the heart. That is, the systemic circulation ‘downloads’ oxygen from red blood cells to the tissues, and the pulmonary circulation ‘uploads’ oxygen to red blood cells as they transit through the lungs.

In the systemic circulation, the left ventricle of the heart acts as a pump, sending blood laden with oxygen out through the systemic vasculature. The systemic vasculature is functionally divided into the arteries, the arterioles, the capillaries, and the venous system. In the arteries, flow is rapid and blood pressure is preserved. The arterioles convert high pressure pulsatile flow into low-pressure, slow, near-continuous flow. In the capillaries, where flow is slow and near-continuous and pressure is low, oxygen is unloaded (from haemoglobin, which is a protein in the blood) and carbon dioxide is loaded (onto haemoglobin), and the venous system transports the carbon dioxide back to the heart.

In the systemic circulation, four elements which contribute to perfusion are: (1) blood volume, which functionally consists of preload and intravascular volume, (2) heart contractility, (3) the resistance/ventricular afterload provided by the systemic vasculature, and (4) heart rate and rhythm. ‘Preload’ refers to the volume of blood that distends the left ventricle before it contracts (the volume of blood inside the left ventricle), ‘intravascular volume’ refers to the blood volume within the systemic vasculature, ‘heart contractility’ refers to the function of the heart as a muscle, and the resistance of the systemic vasculature (or ‘ventricular afterload’) refers to the capacity of the tissues to vary vasomotor tone to regulate blood flow. Heart rate refers to the speed at which the heart beats, and rhythm to the regularity of this beating.

In the systemic circulation, the downloading of oxygen to the tissues cannot itself be measured, but there are measurable elements from which the performance of the systemic circulation can be inferred. Blood pressure can be measured and is the product of preload, heart contractility, and the resistance/afterload of the systemic vasculature. The mean arteriovenous pressure gradient is one measure of blood pressure and is calculated as (MAP−CVP) where MAP is mean arterial pressure and CVP is central venous pressure. Preload and heart contractility together generate left ventricle stroke volume (SV), and SV can be estimated. Resistance/ventricular afterload may be estimated, such as by being calculated from measurements of blood pressure and estimates of blood flow or SV (e.g. systemic vascular resistance abbreviated as SVR, systemic elastance abbreviated as Esys, effective arterial elastance abbreviated as Ea). Heart rate (HR) and rhythm can be measured. Blood flow (typically defined as cardiac output, that is CO) is the product of preload, heart contractility, and heart rate, and can be estimated. These measurable elements or parameters are used to understand the performance of the systemic circulation. Intravascular volume cannot be measured or reliably estimated.

SVR is the most commonly-used measure of vascular tone and is measured over successive cardiac cycles. Esys is an alternative beat-to-beat measure of vascular tone and there is early evidence of its value as a more granular measure of vascular tone. That is, SVR (in mmHg/L/min) can be expressed in terms of Esys (mmHg/L) and HR such that:

$$S V {R}_{i} = \frac{{E}_{s y {s}_{t}}}{H {R}_{i}}$$

The inventor has observed that where Esys is changing, this change is often offset by a change in HR so that SVR will remain relatively steady. Thus, for example if Esys increases significantly but heart rate also increases significantly, SVR may indicate normal vascular tone while Esys indicates marked vasoconstriction. The inventor has observed this phenomenon during haemorrhage and acute atrial fibrillation. His thesis “The pressure field defined by stroke volume and systemic elastance as a novel paradigm for perfusion management of the circulatory system” (https://doi.org/10_25949/13909577_v1) outlines case studies of this phenomenon during acute atrial fibrillation.

Further, there is no clear consensus among anaesthetists and intensivists regarding optimal targets for haemodynamic parameters in patients undergoing surgery or being cared for in the intensive care unit (ICU). A wide range of targets for some of the above parameters, and derivatives of these parameters including Eadyn and dp/dTmax, have been proposed.

However, ‘personalised haemodynamics’ is one management paradigm for which there is emerging evidence of a clinical benefit. Personalised haemodynamics is based on the recognition that population-wide reference ranges do not reflect the diversity of physiology seen in perioperative medicine. According to this paradigm, management decisions are made with reference to the ‘normal haemodynamic values’ of a patient. The paradigm has most commonly been applied with reference to a patient's blood pressure and CO. However, the inventor's research has led to the hypothesis that this paradigm may be of greater benefit when a broader set of haemodynamic parameters (including Esys and variants of it such as Ea) are also accounted for.

In the pulmonary circulation, blood only travels a short distance and so the work required to be performed by the right ventricle is much lower than the work performed by the left ventricle. The performance of the pulmonary circulation can be assessed using equivalent parameters to those used to assess the performance of the systemic circulation. The pulmonary perfusion pressure (PPP) results from the interaction between the right ventricle and the pulmonary arteries, arterioles, capillaries and veins. For every pressure wave generated in the pulmonary circulation, the contribution of the right ventricle is the right ventricular stroke volume (RVSV), and the contribution of the pulmonary vasculature is the pulmonary elastance (Ep). PPP is the difference between the right ventricular outflow pressure (mean pulmonary artery pressure) and the left atrial inflow pressure (estimated using pulmonary capillary wedge pressure). SV may be approximated by RVSV.

Pathological processes in the pulmonary circulation (for example, right heart failure or pulmonary hypertension) are different to pathological processes in the systemic circulation. Both are of relevance in cardiovascular medicine, although the systemic circulation is of primary importance.

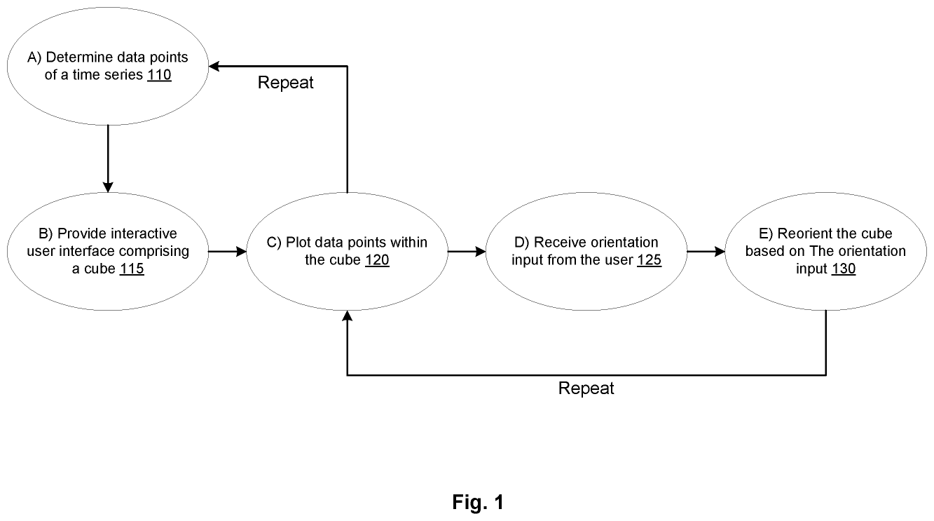

FIG. 1 is a process flow diagram showing a high-level overview of a method 100 of generating and providing an interactive 3D user interface, according to some embodiments. In particular, and with reference to FIG. 1, the present disclosures relate to methods for generating interactive 3D user interfaces of haemodynamic performance data of a subject.

At step 110, one or more data points are determined. The data points may comprise a first variable, a second variable and/or a third variable. The first, second and third variables are typically different from one another. In some embodiments, the data points may be determined from biometric data collected from one or more monitoring devices configured to record biometric data of a subject, such as one or more sensors placed on the skin of the subject, or inserted into the subject, such as into a vein, or into the heart of the subject. Such monitoring devices may include vital signs monitors and/or a haemodynamic monitors. The monitoring device(s) may be configured to record the biometric measurements and pass them to a system or device executing method 100 or the monitoring device(s) may be configured to perform calculations based on the measurements to determine indirectly measured biometric data derived from the measurements, and pass the indirectly measured biometric data and/or the biometric measurements to the system or device.

The first, second and/or third variables may be one of: systemic vascular resistance (SVR), total peripheral resistance (TPR), heart rate (HR), mean arterial pressure (MAP), stroke volume (SV), systemic perfusion pressure (SPP), systemic elastance (Esys), effective arterial elastance (Ea), cardiac cycle time (CC), cardiac output (CO), stroke volume index (SVI), cardiac index (CI), right ventricle stroke volume (RVSV), pulmonary perfusion pressure (PPP), pulmonary elastance (Ep), right ventricle cardiac output (RVCO), right ventricle stroke volume index (RVSVI), right ventricle cardiac index (RVCI), indexed pulmonary elastance (EpI), left atrial pressure (LAP), pulmonary capillary wedge pressure (PCWP), mean pulmonary arterial pressure (MPAP), indexed systemic vascular resistance (SVRI), indexed systemic elastance (EsysI), indexed effective arterial elastance (EaI), indexed total peripheral resistance (TPRI), and/or indexed pulmonary vascular resistance (PVRI), for example. The index or indexed variables are indexed based on a body surface area (BSA) of the subject. In some embodiments, HR and CC may be used interchangeably; in other words, either HR or CC may be used as one of the variables of the data points.

In some embodiments, the data points may be plotted over time, such as evenly over time. However, heart rate and/or CC is never perfectly regular and accordingly in some embodiments the data points may be plotted at an interval which reflects CC. The cardiac cycle comprises all of the physiological events associated with a single heartbeat, including electrical events, mechanical events (pressures and volumes), and heart sounds. A single cardiac cycle may comprise one full set of physiological events associated with a single heartbeat, which may occur slightly before and/or slightly after the actual heart beat they are associated with.

At step 115, an interactive graphical user interface is provided on a display device. The interactive user interface comprises a visual representation (e.g., cube) that defines a 3D space. The data points may be plotted based on the values of the first, second and third variables of each of the data points, relative to the respective edge of the cube indicative of each of the variables.

At step 120, the data points are plotted within the 3D space defined by the visual representation of the interactive user interface such that the first, second, and third variables and/or the resulting health measures, indicative of health information of the subject may be interacted with and may be subsequently interpreted by a clinical physician or other clinical professional. The interactive user interface may be presented via a computing device, such as a personal computer, tablet computer, smart phone and/or any other type of display technology. At least three edges of the visual representation may each be indicative of one of the first, second or third variables determined at step 115. At least three of the faces of the visual representation may be indicative of a relationship between two of the first, second and third variables.

In some embodiments, steps 110, 115, and 120 may be performed iteratively. For example, when the present method 100 is performed ‘live’, when biometric data is being provided in real time for determining the data points in real time, such as during surgery, the user interface may be continuously determining the data points, and plotting the data points within the 3D space. In other embodiments, method 100 may be performed after the surgery has been conducted, for example to study what transpired during the surgery. In this case, the biometric data from which the first, second and/or third variables are derived may be retrieved from data storage.

At step 125, the user interface may receive an orientation input from a user to change the orientation of the visual representation in some way. The orientation input received from the user would not be used to change or alter the values of the first, second and/or third variables. The input may be received via any type of input method, such as a touch interface, buttons, keys, computer mouse, and/or any other form of input device or method.

Orientation of the cube may comprise the viewing angle of the visual representation, the rotation of the visual representation in an axis or combination of axes, and/or the level of zoom towards or away from the visual representation. An orientation input may comprise a change to any one or more aspects of the presentation of the cube.

At step 130, in response to receiving the orientation input from the user, the visual representation may be caused to change its orientation as presented by the interactive user interface.

In some embodiments, for example, when the data points are displayed in the 3D space of the visual representation, the user may swipe or otherwise interact with a display or screen showing the interactive graphical user interface to align one face of the visual representation to be substantially parallel to the screen, at which time, the visual representation may transform or otherwise alter to become a two-dimensional graph. For example, the cube or graph may comprise one or more new features, such as reference lines and/or lines demarcating regions that indicate data points indicative of a good or a normal health status of the subject, and/or an adverse health status of the subject.

In some embodiments, steps 120, 125 and 130 may be performed iteratively such that a user may input multiple, for example, subsequent orientation inputs, to change the visual representation displayed on the interactive user interface at will. For example, if the user has manipulated the interactive user interface or visual representation to show the two-dimensional representation described above, the user may interact with the user interface to return to the visual representation.

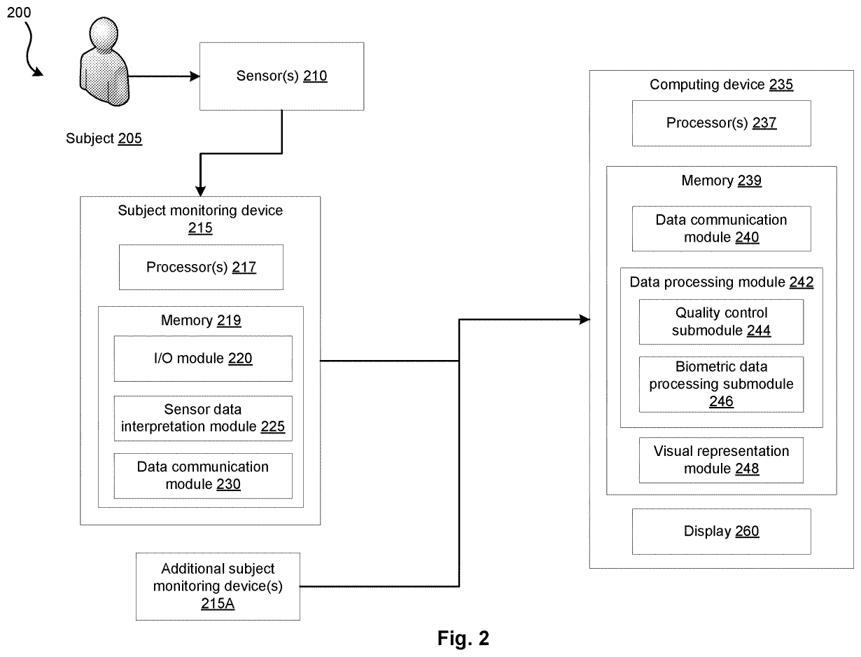

FIG. 2 is a block diagram of system 200 for generating and providing an interactive 3D user interface, according to some embodiments. The system 200 comprises sensor(s) 210, subject monitoring device 215, additional subject monitoring device(s) 215A, and/or computing device 235.

The subject monitoring device 215 is configured to read, sense, observe or otherwise collect readings from a subject 205, for example, via sensor(s) 210. The subject monitoring device 215 may be a heart rate monitor, blood pressure monitor, oxygen saturation monitor, respiratory rate monitor, temperature monitor, ECG, haemodynamic monitor and/or an end-tidal carbon dioxide (ETCO2) monitor.

The sensor(s) 210 may be airflow sensors, pressure sensors, oxygen sensors, temperature sensors, magnetic sensors, thermistors, force sensors, position sensors and/or temperature/humidity sensors. In other words, sensor(s) 210 are configured to read, detect and/or measure one or more physical, chemical and/or biological properties of a subject, and transfer a representation of those measured physical properties to subject monitoring device 215 to monitor and/or record the health status of the subject 205.

Subject monitoring device 215 may comprise one or more processor(s) 217 and memory 219 storing instructions (e.g. program code) which when executed by the processor(s) 217 causes the subject monitoring device 215 to record, communicate and/or interpret biometric data from the subject 205, via sensor(s) 210. In some embodiments, subject monitoring device 215 may be in communication with computing device 235 to perform the presently disclosed methods.

The processor(s) 217 may comprise one or more microprocessors, central processing units (CPUs), application specific instruction set processors (ASIPs), application specific integrated circuits (ASICs) or other processors capable of reading and executing instruction code.

Memory 219 may comprise one or more volatile or non-volatile memory types. For example, memory 219 may comprise one or more of random access memory (RAM), read-only memory (ROM), electrically erasable programmable read-only memory (EEPROM) or flash memory. Memory 219 is configured to store program code accessible by the processor(s) 217. The program code comprises executable program code modules. In other words, memory 219 is configured to store executable code modules configured to be executable by the processor(s) 217. The executable code modules, when executed by the processor(s) 217 cause the subject monitoring device 215 to monitor the subject and/or communicate subject data to computing device 235, as described in more detail below. For example, memory 219 may comprise I/O module 220, signal interpretation module 225 and/or data communication module 230, for example.

The I/O module 220 is configured to interface and/or communicate with sensor(s) 210 to receive readings and/or measurements from the sensor(s) 210. In some embodiments, I/O module is configured to receive sensor data, such as the presence and/or absence of or changes in electrical impulses as induced by the subject's heartbeat, and/or changes in a subject's blood pressure, as measured by the sensor(s) 210. I/O module 220, in some embodiments, is configured to interpret the electrical impulses and/or pressure to convert them into a representation, data type or otherwise format that is usable by sensor data interpretation module 225. I/O module is configured to communicate the received sensor data to sensor data interpretation module 225 for further processing and/or interpret.

Sensor data interpretation module 225 is configured to calculate and/or determine, based on the received sensor data, biometric data indicative of the health information, health status and/or status of biological processes of the subject 205. For example, in some embodiments, the sensor data interpretation module 225 is configured to convert blood pressure measurements into an estimate of stroke volume. Subsequent to the sensor data being interpreted, and the generation of the biometric data by the sensor data interpretation module 225, data communication module 230 communicates the biometric data to computing device 235, for it to be further processed. Data communication module 230 may be in communication with computing device 235 over a wired connection such as USB-A, USB-C, Firewire, micro-USB or any other type of suitable wired connection. In some embodiments, subject monitoring device 215 and computing device 235 are in communication by a wireless connection such as Wi-Fi, Blue Tooth, Zigbee, RFID, 6LoWPAN, or near field communication) NFC or any other type of suitable wireless communication protocol.

Additional subject monitoring device(s) 215A are configured to monitor the same, similar and/or different subject biometric phenomena and/or processes as subject monitoring device 210. The biometric data recorded, sensed or otherwise collected by the additional subject monitoring device(s) 215A may be used by the system 200 to determine one or more data points comprising a first, second and/or third variable or measure indicative of health information of a subject based on the respective biometric data in isolation, or in combination with biometric data collected by subject monitoring device 215 and/or any one or more other additional subject monitoring device(s) 215A.

Computing device 235 may comprise a mobile or handheld computing device such as a smartphone or tablet, a laptop, or a PC, and may, in some embodiments, comprise multiple computing devices. Computing device 235 may comprise one or more processor(s) 237 and memory 239 storing instructions (e.g. program code) which when executed by the processor(s) 237 causes the computing device 235 to generate and provide an interactive 3D user interface. In some embodiments, computing device 235 may be in communication with subject monitoring device 215 and/or additional subject monitoring device(s) 215A to perform the presently disclosed methods.

The processor(s) 237 may comprise one or more microprocessors, central processing units (CPUs), application specific instruction set processors (ASIPs), application specific integrated circuits (ASICs) or other processors capable of reading and executing instruction code.

Memory 239 may comprise one or more volatile or non-volatile memory types. For example, memory 239 may comprise one or more of random access memory (RAM), read-only memory (ROM), electrically erasable programmable read-only memory (EEPROM) or flash memory. Memory 239 is configured to store program code accessible by the processor(s) 237. The program code comprises executable program code modules. In other words, memory 239 is configured to store executable code modules configured to be executable by the processor(s) 237. The executable code modules, when executed by the processor(s) 237 cause the computing device 235 to generate and provide an interactive 3D user interface, as described in more detail below. For example, memory 239 may comprise data communication module 240, data processing module 242 and visual representation module 248, for example.

In some embodiments, computing device 235 may comprise one or more display, such as an LED or LCD screen for displaying visual representations of subject data and/or subject health metrics. The display of computing device 235 may, in some embodiments, be a touch control display, usable with one or more of a user's digits and/or a stylus. Computing device 235 may comprise one or more peripherals, such as a keyboard, mouse, touch pad, joystick, button array and/or microphone.

The data communication module 240 may be configured to receive biometric data from subject monitoring device 215. In some embodiments, subject monitoring device 215 and computing device 235 may be in communication over a wired connection such as USB-A, USB-C, Firewire, micro-USB or any other type of suitable wired connection. In some embodiments, subject monitoring device 215 and computing device 235 are connected by a wireless connection such as Wi-Fi, Blue Tooth, Zigbee, RFID, 6LoWPAN, or near field communication) NFC or any other type of suitable wireless communication protocol. The data communication module 240 may, in some embodiments, be configured to provide the biometric data received from the subject monitoring device to one or more modules of the computing device 235, such as data processing module 242 and/or visual representation module 248, for example.

Data processing module 242 may comprise quality control submodule 244 and/or biometric data processing submodule 246 and be configured to receive the biometric data from data communication module 240 for processing. In some embodiments quality control submodule 244 may receive the biometric data from data communication module 240, to perform data checking and/or sanitisation. For example, quality control submodule 244 may be configured to remove NULL data points, and/or any data points that do not satisfy one or more quality metrics, such as if a data point lies outside a certain acceptable range of possible values. In some embodiments, quality control submodule 244 may be configured to alert a user of the system 200 if the biometric data comprises more than a certain number and/or percentage of bad data points, such as by causing a warning to be displayed on display 260.

In some embodiments, the biometric data processing submodule 246 is configured to process and/or interpret the biometric data to calculate, generate or otherwise determine one or more data points comprising a first variable, a second variable or a third variable indicative of health information of the subject 205. In some embodiments, the one or more data points and/or the first, second and/or third variables may be indicative of the subject's haemodynamic performance.

The biometric data processing submodule 246 may be configured to determine one or more of the following variables or measures: systemic vascular resistance (SVR), total peripheral resistance (TPR), heart rate (HR), central venous pressure (CVP), mean arterial pressure (MAP), stroke volume (SV), systemic perfusion pressure (SPP), systemic elastance (Esys), effective arterial elastance (Ea), and/or cardiac output (CO), for example. Cardiac cycle (CC) may, in some embodiments, be determined by biometric data processing submodule 246. In some embodiments, the determination process may comprise using one or more mathematical equations that describe a biological process and/or health status of the subject, such as performance of the systemic circulation, for example:

$$S P P = C O \times S VR ,$$

$$MAP = CO \times T PR ,$$

$$CO = HR \times SV ,$$

$$SPP = MA P - C VP ,$$

$$SPP = HR \times S V \times S VR ,$$

$$SPP = HR \times S V \times \frac{E s y s}{H R} ,$$

$$SVR = \frac{E s y s}{H R} ,$$

$$SPP = S V \times Esys , and$$

$$MAP = SV \times E a .$$

As shown in the above equations, central venous pressure (CVP) may be used to calculate one or more variables or measures. CVP may be determined, assumed, inferred and/or calculated in various ways. For example, CVP may be estimated based on other qualities of the subject 205, such as age, weight, current health status and/or medical history. CVP, in some embodiments, may be assumed to be zero, functionally making SPP=MAP, Esys=Ea, and SVR=TPR.

The above equations are based on SVR in mmHg/L/min and Esys being expressed in mmHg/L. If SVR is expressed in dynes·sec·cm⁻⁵ then the above equations are adjusted to reflect the relationship SVR=(E_{sys}×80)/HR.

The above equations use HR to express the frequency at which the heart beats. If cardiac cycle time (CC) is used to express the frequency at which the heart beats (in seconds per beat), then the above equations are adjusted according to the relationship

$$C C = \frac{1}{H R} \times 60 .$$

In some embodiments, one or more of the above-mentioned variables or measures may further be indexed to the body surface area (BSA) of the subject 205 to create the following variables: indexed systemic vascular resistance (SVRI), indexed stroke volume (SVI), indexed cardiac output (CI), indexed systemic elastance (EsysI), indexed effective arterial elastance (EaI) and/or indexed total peripheral resistance (TPRI). The indexed variables may be determined by dividing a variable by the BSA of the subject 205.

According to some embodiments of the present disclosures, biometric data processing submodule 246 may be configured to determine one or more variables or measures, such as left ventricular stroke work (LVSW), rate pressure product (RPP), and cardiac power (CP).

LVSW may be determined using the equations:

$$LVSW = ( M A P - C V P ) \times SV ; and / or$$

$$LVSW = ( MA P - L V E D P ) \times S V ,$$

where LVEDP is left ventricular end-diastolic pressure. LVSW may also be indexed to BSA by dividing LVSW by BSA.

CP may be determined using the equations:

$$C P = L V S W \times HR ;$$

$$CP = S P P \times CO ; and / or$$

$$CP = ( M A P - L V E D P ) \times C O .$$

CP may be indexed to CPI by dividing CP by BSA, or using the equations:

$$CPI = LVSWI \times HR ;$$

$$CPI = SPP \times SVI \times HR ; or$$

$$CPI = SPP \times CI ;$$

RPP may be determined using the equation RPP=SPP×HR.

According to some embodiments, the biometric data processing submodule 246 may be configured to determine one or more of the following variables or measures: mean pulmonary arterial pressure (MPAP), pulmonary capillary wedge pressure (PCWP), left atrial pressure (LAP), pulmonary perfusion pressure (PPP), pulmonary vascular resistance (PVR), right ventricle cardiac output (RVCO), right ventricle stroke volume (RVSV), pulmonary elastance (Ep), and/or heart rate (HR). Cardiac cycle (CC) may, in some embodiments be determined by biometric data processing submodule 246. In some embodiments, the determination process may comprise using one or more mathematical equations that describe a biological process and/or health status of the subject, such as performance of the pulmonary circulation, for example:

$$P P P = M P A P - P C WP ,$$

$$PVR = \frac{( M P A P - P C W P )}{R V C O} ,$$

$$Ep = \frac{( M P A P - P C W P )}{R V S V} , and$$

$$RVCO = R V S V \times H R .$$

RVSV may be approximated by SV, RVCO may be approximated by CO, and PCWP is an approximation of LAP.

The above equations are based on PVR in mmHg/L/min and Ep being expressed in mmHg/L. If PVR is expressed in dynes·sec·cm⁻⁵ then the above equations must be appropriately adjusted to reflect the relationship PVR=(Eₚ×80)/HR.

The above equations use HR to express the frequency at which the heart beats. If cardiac cycle time (CC) is used to express the frequency at which the heart beats, then the above equations are adjusted according to the relationship

$$C C = \frac{1}{H R} \times 60 .$$

According to some embodiments of the present disclosures, biometric data processing submodule 246 may be configured to determine one or more variables or measures, such as right ventricle stroke work (RVSW), right ventricle power (RVP), right ventricle power index (RVPI), right ventricle stroke work index (RVSWI), right ventricle stroke volume index (RVSVI), right ventricle cardiac index (RVCI), right ventricle rate pressure product (RVRPP), and indexed pulmonary elastance (EpI).

RVSW may be determined using the equation RVSW=PPP×RVSV. RVSW may also be indexed to BSA by dividing RVSW by BSA.

RVP may be determined using the equation RVP=PPP×RVCO.

RVP may be indexed to RVPI by dividing RVP by BSA, or RVPI may be determined using the equation RVPI=PPP×RVCI.

RVRPP may be determined using the equation RVRPP=PPP×HR.

RVSV may be indexed to BSA by dividing RVSV by BSA.

RVCO may be indexed to BSA by dividing RVCO by BSA.

Ep may be indexed to BSA by multiplying Ep by BSA.

However, it will be appreciated that it is possible to determine or calculate the one or more variables or measures relating to the left part of the heart, such as SPP, CP and LVSW, without determining or using values for CVP. Similarly, it will also be appreciated that it is possible to determine or calculate the one or more variables or measures relating to the right part of the heart, such as PPP or Ep or PVR, without determining or using values relating to downstream pressure, such as LAP or PCWP.

The visual generation module 248 is configured to generate and display the interactive user interface, and to plot the data points determined by the biometric data processing submodule 246. In some embodiments, the interactive user interface, the visual representation and the data points plotted in the 3D space of the visual representation are indicative of an extent, performance, and/or rate of a biological process of the subject. The visual representation module 248, in some embodiments, is configured to change, reorient and/or adapt the visual representation in relation to orientation inputs from a user. In some embodiments, the interactive user interface comprising the visual representation is displayed on/by a display 260. In some embodiments, one or more edges of the visual representation is an axis indicative of and/or associated with one of the first, second or third variables of the data points, such as heart rate or stroke volume, for example. At least one face of the visual representation and in some embodiments more than two faces of the visual representation may be indicative of a relationship between two of the first, second or third variables. For example, a face of the visual representation may depict a two-dimensional graph (such as a line graph or scatter plot) comprising a first edge, or axis, indicative of a first health variable (e.g. the first variable), such as stroke volume, and a second edge, or axis, indicative of a second heath variable (e.g. the second variable), such as heart rate. In some embodiments, one or more edges of the visual representation may be indicative of time or another non-medical variable that other variables indicative of a subject's health information may be measured over. Additionally, or alternatively, the order in which the data points are plotted in the 3D space of the visual representation may be indicative of time, or otherwise a sequence of the data points, based, for example on the order in which biometric data was received and the data points were determined.

Subsequent to receiving an orientation input from user interacting with the display 260, visual representation module 248 may be configured to transform and/or adapt all or some of the visual representation, based at least partially on the orientation input. For example, the visual generation module 248 may be caused to do one or a combination of two or more of zoom in, zoom out, and/or rotate the visual representation around any one or more axes of rotation. In some embodiments, the visual representation may be transformed into a new representation, for example if the visual representation is rotated to align one face of the visual representation substantially parallel to the surface of the display, the visual representation may be transformed to be a two-dimensional graph with a subset of the variables of the one or more data points, for example, only the first and second variables. In this way, the interactive user interface and the data points displayed within the 3D space of the visual representation may be more intuitive and more effectively present complex and/or large numbers of data points and/or the relationships between the variables associated with the data points.

Display 260 is configured to display the interactive user interface comprising the visual representation (such as a cube) and one or more, or a combination of two or more of data read outs, buttons, switches, sliders and/or menus. The values of the first second and/or third variables or measures, and/or any mean or median or other statistically derived values may be presented on display 260 via one or more readouts. Display 260 may be an LCD display, LED display, CTR display, vacuum fluorescent display, electroluminescent (ELD) display, a plasma (PDP) display or a projector display, for example. Display 260 may be integrated with computing device 235, such as the screen of a smart phone, a tablet computer, laptop or a vital sign monitoring computing device. Display 260 may a peripheral, connected to and/or operable in conjunction with computing device 235, such as a display monitor, TV screen or a projector, connected by a wired or wireless connection to computing device 235.

In some embodiments, display 260 may be a touch screen display and may be configured to detect and interpret touch interactions from a user's hand and/or fingers. Display 260 may also be configured to communicate the touch interactions of the user to the visual representation module 248, to allow the visual representation module 248 to orient, transform or adapt the visual representation based at least in part on the touch interaction.

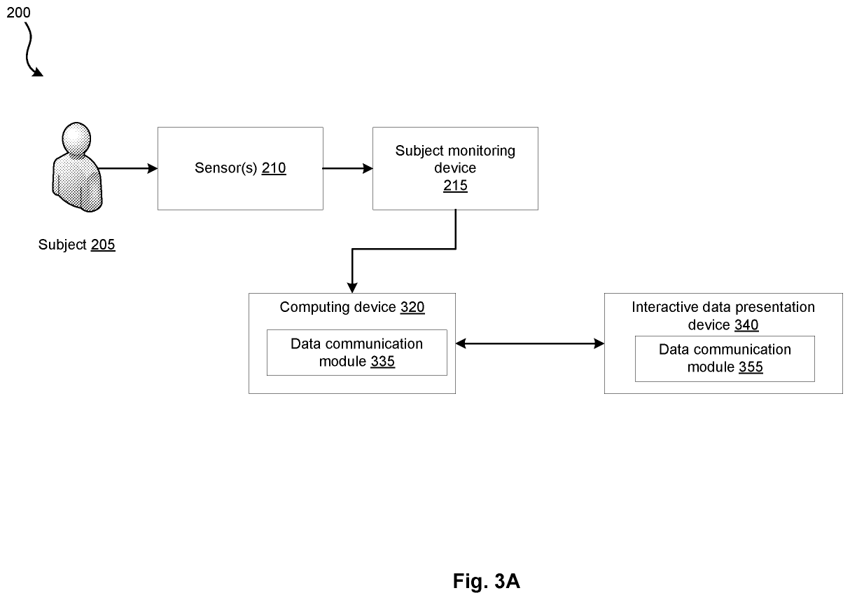

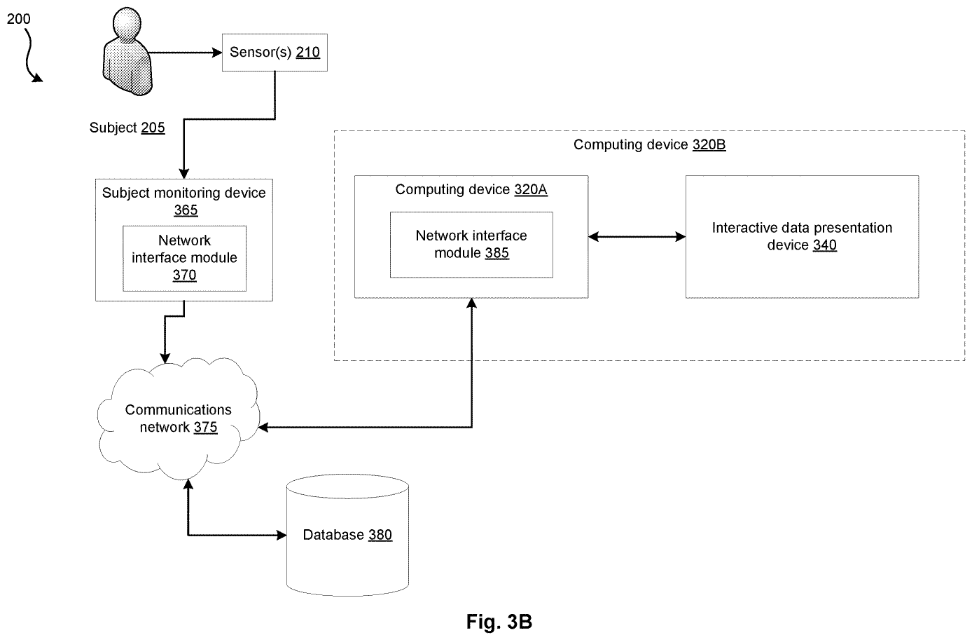

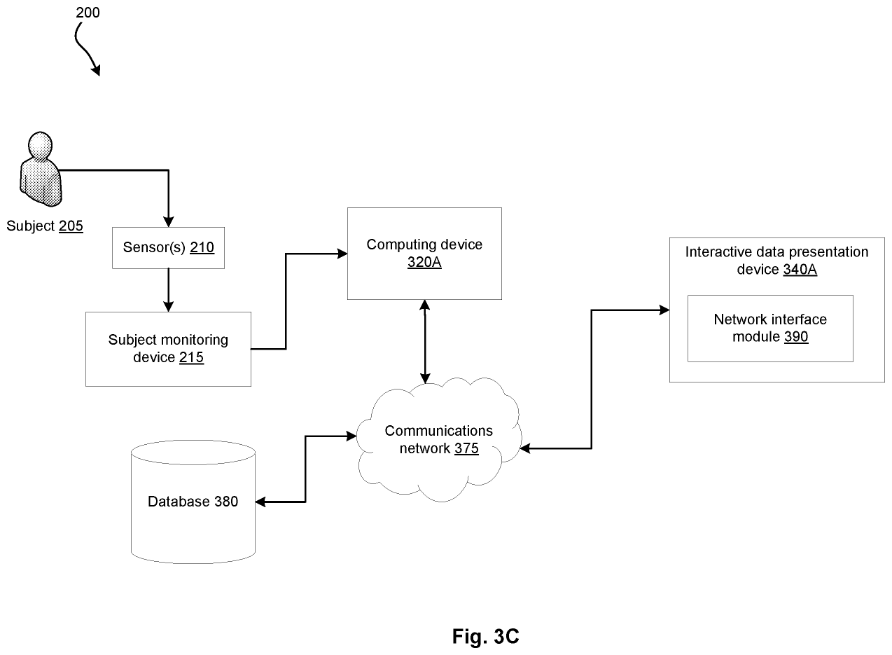

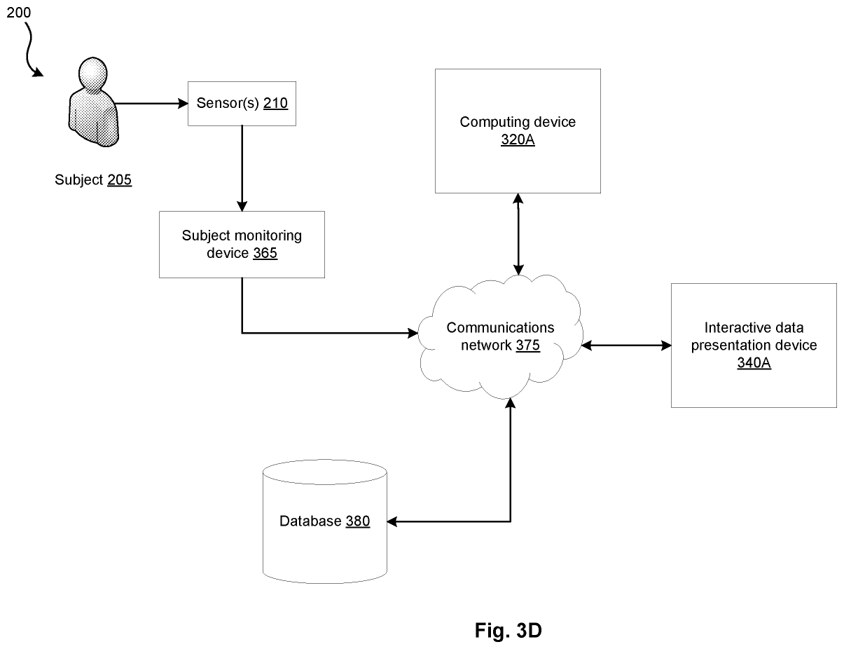

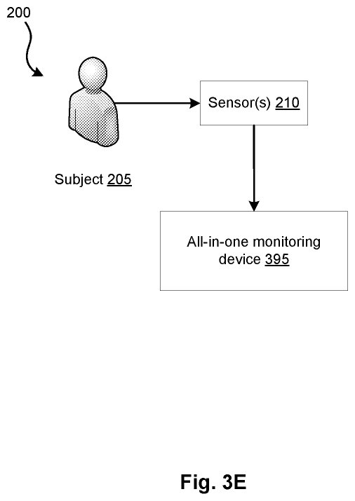

FIG. 3A to FIG. 3E are block diagrams of the system for presenting biometric data of FIG. 2, according to alternate embodiments. It will be understood by the person skilled in the art that the various components of the system 200, as described by FIG. 2 and associated description, may be arranged in various different configurations without departing from the broad general scope of the present disclosure.

In the embodiment depicted by FIG. 3A, display 260 may not be integrated into computing device and instead may be a peripheral operably connected, via wired or wireless connection, to the computing device. In some embodiments, system 200 may comprise computing device 320 and interactive data presentation device 340.

Computing device 320 is configured to receive the biometric data from the subject monitoring device 215, process the biometric data to determine first, second, and third variables of the one or more data points and generate and provide the visual representation as part of the interactive user interface, and may function in a similar way to computing device 235 as described above. Computing device 320 may comprise one or more of the same modules as computing device 235 as depicted in FIG. 2, as well as data communication module 355, configured to communicate the instructions to render the interactive user interface and the one or more data points to one or more interactive data presentation device 340 and/or receive from interactive data presentation device 340 indications as to the occurrence and/or nature of an orientation input from a user of the interactive data presentation device 340. In some embodiments, computing device 320 may be a laptop, desktop, tablet computer, smart phone or server.

Interactive data presentation device 340 is configured to display the interactive user interface, the visual representation and the data points plotted in the 3D space of the visual representation and/or receive input from the user. Interactive data presentation device 340 may function similarly to display 260 as described above. Interactive data presentation device 340 may comprise, in some embodiments, data communication module 355. Data communication module 355 is configured to receive the instructions to render the interactive user interface and the one or more data points from computing device and/or communicate orientation inputs from the user to computing device 320 for use in orientating, adapting or transforming the visual representation based in part on the user interaction. In some embodiments, the interactive data presentation device 340 may be a computing device such as a smart phone, tablet, laptop or desktop computer, or in some embodiments, may be a display, such as a monitor or television.

The embodiment of FIG. 3A may comprise one or more additional interactive data presentation device (not shown) in simultaneous communication with computing device 320. For example, during a surgical procedure, computing device 320 may be in communication with one or more subject monitoring device 215 connected to the subject 205 undergoing the surgery procedure. Computing device 320 may also be in communication with a number of interactive data presentation devices 340 positioned at different locations or stations in the operating theatre. In this way, the system 200 may provide a number of participants in the surgery at different stations, such as a surgeon, an anaesthetist, and/or one or more assisting professionals such as surgical nurses, with the interactive user interface without a need for the processing to be performed at each station, potentially saving space in an already crowded operating theatre by reducing the number of computing devices at each station of the operating theatre. The one or more interactive data presentation devices 340 may be configured to display the visual representation in different orientations based upon the orientation inputs received at the particular interactive data presentation devices 340.

According to the embodiment of FIG. 3B, the system 200 may be in communication over a communications network 375. The embodiment of FIG. 3B may comprise subject monitoring device 365, which may comprise one or more of the same modules and be configured to perform the same functions as subject monitoring device 215 as depicted in FIG. 2. Subject monitoring device 365 may comprise network interface module 370 for communicating via communications network 375 with computing device 320A, 320B and/or database 380, to communicate the biometric data.

The communications network 375 may include, for example, at least a portion of one or more networks having one or more nodes that transmit, receive, forward, generate, buffer, store, route, switch, process, or a combination thereof, etc. one or more messages, packets, signals, some combination thereof, or so forth. The communications network 375 may include, for example, one or more of: a wireless network, a wired network, an internet, an intranet, a public network, a packet-switched network, a circuit-switched network, an ad hoc network, an infrastructure network, a public-switched telephone network (PSTN), a cable network, a cellular network, a satellite network, a fibre-optic network, some combination thereof, or so forth.

The database 380, which may form part of or be local to the system 200, or may be remote from and accessible to the system 200, for example, via the communications network 375. The database 380 may be configured to store data associated with the system 200. The database 380 may be a centralised database. The database 380 may be a mutable data structure. The database 380 may be a shared data structure. The database 380 may be a data structure supported by database systems such as one or more of PostgreSQL, MongoDB, and/or ElasticSearch. The database 380 may be configured to store a current state of information or current values associated with various attributes (e.g., “current knowledge”).

In some embodiments, computing device 320A may comprise one or more of the same modules and be configured to perform the same functions as computing device 235, 320. Computing device 320A may comprise network interface module 385 configured to communicate with subject monitoring device 365, to receive the biometric data. For example, and/or database 380, to store or retrieve data stored in database 380, via communications network 375. Computing device 320A may also be in communication with interactive data presentation device 340 for displaying the interactive user interface and the data points plotted in the 3D space of the visual representation. In some embodiments, computing device 320A and interactive data presentation device 340 may be different devices in communication with each other, or they may be a single device, such as computing device 320B.

It is envisioned the system 200 as depicted in FIG. 3B may be distributed throughout a location, such as between a surgery theatre and one or more observation rooms or wards. The embodiment of FIG. 3B may also be distributed between multiple locations, for example, between two different hospitals, wherein a clinical professional may be able to observe the status of a subject, such as during a surgery, or engage in subject analysis, via the interactive user interface and data points plotted in the 3D space of the visual representation. In this way, the present disclosures may enable clinical professionals who are not co-located to monitor and/or analyse a subject and this may result in more positive health outcomes for the subject.

FIG. 3C depicts an embodiment of the system 200 wherein the interactive data presentation device 340A may not be co-located with the subject monitoring device 215 and computing device 320A. For example, during a surgery, subject sensing data may be collected by sensor(s) 210 and biometric data may be determined by subject monitoring device 215. The computing device 320A, co-located with the subject monitoring device 215 may process the biometric data to determine the one or more data points and provide the interactive user interface comprising the visual representation. These components and processes may all take place at one location, such as in a surgical theatre wherein a procedure is being performed. The interactive user interface and the one or more data points may then be communicated to interactive data presentation device 340A and any additional interactive data presentation devices (not shown), for presentation to one or more observers, who may be clinical experts whose assistance is being sought.

Interactive data presentation device 340A may be a laptop computer, desktop computer, smart phone, tablet, monitor, TV screen or projector. Interactive data presentation device 340A may comprise one or more of the same modules and be configured to perform the same functions as interactive data presentation device 340 and may comprise network interface module 390 configured to receive instructions to render the interactive user interface and the data points to be plotted in the 3D space of the visual representation from computing device 320A via communications network 375. Interactive data presentation device 340A may also be configured to communicate orientation inputs to computing device 320A via communications network 375.

The embodiment of FIG. 3D is a distributed arrangement of the system 200 wherein the subject monitoring device 365 and the sensor(s) 210, computing device 320A and interactive data presentation device 340A may all be located in different locations, or otherwise not in the same room or immediate area, such as an operating theatre. The embodiment of FIG. 3D may allow the collection of biometric data and communicate the biometric data to the computing device 320A, which may be a central server receiving requests for processing data and generating one or more data points comprising the first, second and third variables of the received biometric data. The computing device 320A may then communicate the instructions for rendering the interactive user interface and the one or more data points to be plotted to the interactive data presentation device 340A, via communications network 375. The system 200 as exemplified by FIG. 3D may comprise multiple additional subject monitoring devices (not shown) and/or additional interactive data presentation devices (not shown). In this way, the system 200 may perform the presently disclosed methods without requiring particular hardware to be present and/or installed at any one particular location where subjects may be present. For example, a subject may be able to configure the subject monitoring device 365 at their home for at home monitoring. The collected biometric data may then be stored for later processing in database 380, or forwarded to computing device 320A for immediate processing. Subsequent to processing, the one or more data points and/or the instructions for rendering the interactive user interface comprising the visual representation may be communicated to database 380 for storage, or communicated to interactive presentation device 340A for presentation to a clinical professional at a different location to the subject.

In this way, the embodiment of FIG. 3D may allow for more complete and/or accurate health determinations for subjects regardless of their geographical location and/or the location of clinical professionals.

As depicted in FIG. 3E, the system 200 may comprise all-in-one monitoring device 395. All-in-one monitoring device 395 may comprise one or more of the same modules and be configured to perform the same functions as subject monitoring device 215, and computing device 235, and comprise display 260, as depicted in FIG. 2. All-in-one monitoring device may be configured to receive sensor data from sensor(s) 210, process the sensor data into biometric data, and subsequently process the biometric data to determine the first, second and third variables and generate the instructions for rendering the interactive user interface and subsequently render the user interface for presentation to a user of the system 200.

In some embodiments, such as the embodiments depicted in FIG. 3B, FIG. 3C and/or FIG. 3D, at any point during the disclosed methods, data that is to be processed, processed data and/or the results of processed data including but not limited to the biometric data, the one or more data points comprising the first, second and third variables and/or instructions for rendering the interactive user interface comprising the visual representation determined by computing device 320, 320A, 320B may be communicated to and stored in database 380 for later use or for record keeping purposes. Similarly, computing device 320, 320A, 320B and/or interactive data presentation device 340A may request and/or receive stored data in database 380 to, respectively, process said data or display the data points in the interactive user interface and visual representation.

In some embodiments, such as described in FIGS. 3A and 3E, at any point during the disclosed methods, data that is to be processed, processed data and/or the results of processed data including but not limited to the biometric data, the one or more data points comprising the first, second and third variables and/or instructions for rendering the interactive user interface comprising the visual representation determined by, subject monitoring device 215, computing device 235 and/or all-in-one monitoring device 395, may be stored for future use in one or more memory (not shown) of subject monitoring device 215, computing device 235 and/or all-in-one monitoring device 395.

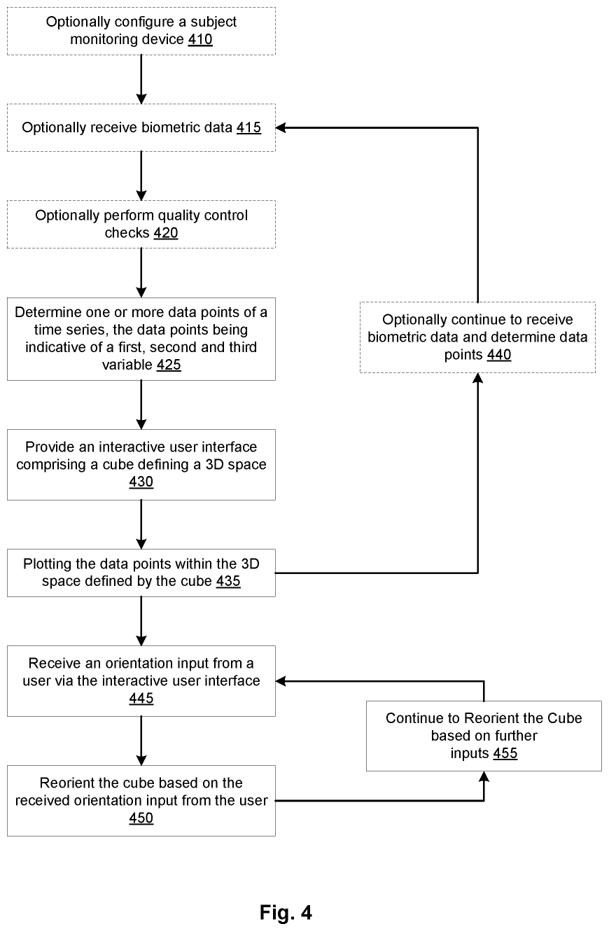

FIG. 4 is a process flow diagram of a method 400 for generating and providing an interactive 3D user interface, according to some embodiments. The method 400 will be described in relation to the embodiment of FIG. 2, however the skilled person would understand that the method may be applied to any and all embodiments of the present systems and methods without departing from the broad general scope of the present disclosures.

Optionally at 410, the subject 205 may be fitted with one or more sensors 210 connected to the subject monitoring device 215 for collecting sensor data to determine biometric data. In some embodiments, the subject 205 may already be fitted with a sensor and/or subject monitoring device 215, and biometric data may already be being collected. In such a case, computing device 235 may simply be connected to the existing subject monitoring device 215 to receive and process the biometric data collected from the subject 205.

In some embodiments, the biometric data of the subject 205 may have previously been collected, and therefore the fitting of a sensor to collect biometric data is not required. In this instance, the computing device 235 may simply be provided with the biometric data that was previously collected by the subject monitoring device 215.

Optionally at 415, the computing device 235 may receive, such as by data communication module 240, the biometric data. In some embodiments, computing device 235 may also have the biometric data, and therefore will not need to receive it. In some embodiments, subject monitoring device 215 may not be configured to determine the biometric data, instead the subject monitoring device may be configured to communicate the sensor data computing device 235 to process the sensor data to determine the biometric data.

The biometric data may be indicative of one or more health variables or measures of a subject 205, a biological process and/or health status of the subject 205. For example, the biometric data may be indicative, for example, of a subject's heart rate over time, or blood pressure over time. In some embodiments, the computing device 235 may receive a plurality of biometric data from one or more subject monitoring device 215 and/or additional subject monitoring device(s) 215A.

The biometric data may be indicative of systemic vascular resistance (SVR), total peripheral resistance (TPR), heart rate (HR), central venous pressure (CVP), mean arterial pressure (MAP), systemic perfusion pressure (SPP), stroke volume (SV), systemic elastance (Esys), effective arterial elastance (Ea), right ventricle stroke volume (RVSV), pulmonary elastance (Ep), pulmonary perfusion pressure (PPP), right ventricle cardiac output (RVCO), total peripheral resistance (TPR), mean pulmonary arterial pressure (MPAP), pulmonary capillary wedge pressure (PCWP), left atrial pressure (LAP), pulmonary vascular resistance (PVR) and/or cardiac output (CO). CC may also be derivable from the biometric data, or in other words, cardiac cycle may be determined from the biometric data. The biometric data may be indicative of indexed stroke volume (SVI), indexed systemic vascular resistance (SVRI), indexed cardiac output (CI), indexed systemic elastance (EsysI), indexed effective arterial elastance (EaI); indexed total peripheral resistance (TPRI), indexed right ventricle stroke volume (RVSVI), indexed right ventricle cardiac output (RVCI), indexed pulmonary vascular resistance (PVRI), and/or indexed pulmonary elastance (EpI). The indexed variables may be indexed based on a body surface area (BSA) of the subject.

Optionally at 420, the quality control submodule 244 of the computing device 215 may perform one or more quality control checks on the biometric data. The quality checks may comprise detecting and removing duplicate data, removing or correcting inaccurate and/or ambiguous data, redact and/or hiding portions of data, checking for and correcting and/or removing inconsistent data, restricting the amount of data, such as for processing capabilities, and/or clean or remove noisy data.

At 425, biometric data processing submodule 246 processes the biometric data to determine one or more data points comprising values for each of a first, second and third variable. The first, second and/or third variables may be indicative of health information of a subject. For example, the health information of the subject may represent perfusion or haemodynamic function in the body of the subject. The first, second and/or third variables may be haemodynamic variables including but not limited to: systemic vascular resistance (SVR), total peripheral resistance (TPR), heart rate (HR), central venous pressure (CVP), mean arterial pressure (MAP), stroke volume (SV), systemic perfusion pressure (SPP), systemic elastance (Esys), effective arterial elastance (Ea), cardiac output (CO), right ventricle stroke volume (RVSV), pulmonary elastance (Ep), pulmonary perfusion pressure (PPP), ventricle cardiac output (RVCO), pulmonary vascular resistance (PVR) and/or cardiac cycle time (CC). The first, second or third variables may be determined using one or more equations indicative of biological processes, for example,

and PPP=MPAP−LAP. In some embodiments, Esys may be expressed as aSVR.

$$SPP = CO \times SVR , MAP = CO \times TPR , CO = HR \times SV , \\ SPP = MAP - CVP , SPP = HR \times SV \times SVR , SPP = HR \times SV \times \frac{{E}_{sys}}{HR} , \\ SVR = \frac{{E}_{sys}}{HR} , SPP = SV \times Esys , PPP = RVCO \times PVR , \\ RVCO = RSVS \times HR , PPP = MPAP - PCWP , \\ PPP = HR \times RVSV \times PVR , PPP = HR \times RVSV \times \frac{Ep}{HR} , \\ PVR = \frac{Ep}{HR} , PPP = RVSV \times EP , MAP = SV \times Ea ;$$

In some embodiments, the processing of the biometric data and/or generation or otherwise determination of the data points and/or the first, second, or third variables may be performed using methods and/or systems described in U.S. Pat. No. 9,173,575 entitled ‘Determining Hemodynamic Performance’ to Stephen Woodford, the contents of which are hereby incorporated by reference.

In some embodiments, the biometric data may be indicative of one or more variables. One or more variables may be estimated or inferred based on one or more of subject 205 age, weight, height, sex, current health status and/or previous medical history and/or may be assumed to be zero. Variables, in some embodiments, may be calculated based on two or more collected, estimated or inferred variables and/or previously determined variables. For example, SPP may be measured from a subject 205, SV may be estimated based on one or more criteria, and Esys subsequently calculated based on SPP and SV.

At 430, visual representation module 248 provides an interactive user interface. In some embodiments, visual representation module 248 may generate one or more data signals to provide the data points to be plotted to display 260 for generating and displaying the data points as part of the interactive user interface. In some embodiments, visual representation submodule 248 may generate instructions to render the interactive user interface communicate these instructions to display 260 for rendering.

The interactive user interface may comprise a visual representation that defines a 3D space or an internal 3D space, for example, a structure or cube or cube structure that defines 3D space or an internal 3D space. The visual representation or cube is comprised of 12 edges, of which at least three of the visual representation's edges are indicative of one of the first, second, or third variables. A first edge of the visual representation is representative of the first variable, a second edge of the visual representation is representative of the second variable, and a third edge of the visual representation is representative of the third variable. A first face of the visual representation defined by the first and second edges depicts a first health measure defined by a function of the first and second variables, a second face of the visual representation defined by the first and third edges depicts a second health measure defined by a function of the first and third variables, and a third face of the visual representation defined by the second and third edges depicts a third health measure defined by a function of the first and third variables. Accordingly, the 3D space constituted by the internal volume of the visual representation is indicative of the relationship between the first, second and third variables plotted therein. In some embodiments, the data points are plotted in an animated fashion, appearing based on the time of recording of the particular data point, in this way time may be represented without being afforded a particular axis, or edge, of the visual representation. The data points may be automatically or manually separated into groupings based on one or more characteristics, such as time of recording, one or more value ranges and/or associated condition of the subject, such as cardiac arrest.

At least three of the faces of the visual representation have a same edge or in other words, share a common edge. For example, a first face may be defined by first and second edges, indicative of first and second variables respectively, a second face may be defined by first and third edges, indicative of first and third variables respectively, and a third face may be defined by second and third edges, indicative of second and third variables respectively. In some embodiments, at least three of the faces of the visual representation constitute a two-dimensional graph showing a health measure as defined by the relationship between two of the first, second or third variables, or otherwise the variables associated with at least two of the edges that form the particular face of the visual representation. For example, a first face of the visual representation may have a first edge that is indicative of the variable SV, and a second edge indicative of the variable Esys, therefore the first face is a two-dimensional graph of the relationship between SV and Esys, or otherwise is indicative of a first health measure SPP. A second face may have a first edge that is indicative of the variable SV and a second edge that is indicative of the variable HR, therefore the second face is a two-dimensional graph of the relationship between SV and HR, or otherwise a second health measure CO. A third face may have a first edge that is indicative of the variable Esys and a second edge that is indicative of the variable HR, therefore the third face is a two-dimensional graph of the relationship between Esys and HR, or otherwise a third health measure SVR. It should be understood the order of which the health measures are discussed and their associated reference numbers, (first, second or third health measure) is simply a naming convention and has no relationship to their nature and/or position on a particular face of the visual representation.

Further examples of variables that may be represented by example graphical user interface 500 and in particular visual representation being cube 510 and 3D space 530, and that have not been exemplified in figures will now be described.

In some embodiments, the first face of the cube may comprise a first edge indicative of the variable SPP and a second edge indicative of the variable SVI. Therefore, the first face is a 2D graph of the relationship between SPP and SVI, or in other words LVSWI. The second face of the cube may comprise a first edge indicative of SVI and a second edge indicative of HR, such that the second face is a 2D graph indicative of the relationship between SVI and HR, or in other words CI. The third face of the cube may comprise a first edge that is indicative of SPP, and a second edge indicative of HR, such that the third face is a 2D graph of the relationship between SPP and HR, or in other words the rate pressure product (RPP). When the first face is indicative of LVSWI, the first face may comprise a first set of reference lines and/or a second set of reference lines, the first set of reference lines being indicative of LVSWI, and/or the second set of reference lines being indicative of EsysI. The second face may comprise a set of reference lines indicative of CI. The third face may comprise a set of reference lines indicative of RPP. Subsequent to two or more data points being plotted in 3D space 530, based on HR, time and/or cardiac cycle, the cube 510 may be indicative of a total of 8 different variables simultaneously.

In some embodiments, the first face of the cube may comprise a first edge indicative of the variable SPP and a second edge indicative of the variable CO, such that the first face is a 2D graph of the relationship between SPP and CO, or in other words CP. The second face may comprise a first edge indicative of CO and second edge indicative of HR, as such the second face is a 2D graph of the relationship between CO and HR, or in other words SV. The third face may comprise a first edge indicative of SPP and a second face indicative of HR, such that the third face is a 2D graph of the relationship between SPP and HR, or in other words RPP. The first face may comprise a first set of reference lines and/or a second set of reference lines. The first set of reference lines may be indicative of CP, and/or the second set of reference lines may be indicative SVR. The second face may comprise a set of reference lines indicative of SV. The third face may comprise a set of reference lines indicative of RPP. Subsequent to two or more data point being plotted in 3D space 530, based on HR, time and/or cardiac cycle, the cube 510 may be indicative of a total of 8 different variables simultaneously.

In some embodiments, the first face of the cube 510 may comprise a first edge indicative of the variable SPP and a second edge indicative of the variable CI, such that the first face is a 2D graph of the relationship between SPP and CI, or in other words CPI. The second face may comprise a first edge indicative of CI and a second edge indicative of HR, such that the second face is a 2D graph of the relationship between CI and HR, or in other words SVI. The third face of cube 510 may comprise a first edge indicative of SPP and a second edge indicative of HR, such that the third face is indicative of the relationship between SPP and HR, or in other words RPP. In some embodiments, the first face may comprise a first set of reference lines and/or a second set of reference lines. The first set of reference lines may be indicative of CPI, and/or the second set of reference lines may be indicative of SVRI. The second face may comprise a set of reference lines indicative of SVI. The third face may comprise a set of reference lines indicative of RPP. Subsequent to two or more data point being plotted in 3D space 530, based on HR, time and/or cardiac cycle, the cube 510 may be indicative of a total of 8 different variables simultaneously.

In some embodiments, the first face of the cube may comprise a first edge indicative of the variable SPP and a second edge indicative of the variable SV, and thus the first face is a 2D graph of the relationship between SPP and SV, or in other words LVSW. The second face of the cube may comprise a first edge indicative of SV and a second edge indicative of HR, and therefore be a 2D graph of the relationship between SV and HR, or otherwise CO. The third face of the cube may comprise a first edge indicative of SPP, and a second edge indicative of HR, and thus be a 2D graph of the relationship between SPP and HR, or otherwise RPP. The first face may comprise a set of first reference lines and/or a set of second reference lines. The set of first reference lines may be indicative of LVSW, and/or the set of second reference lines may be indicative of Esys. The second face may have a set of reference lines indicative of CO. The third face may have a set of reference lines indicative of RPP. Subsequent to two or more data point being plotted in 3D space 530, based on HR, time and/or cardiac cycle, the cube 510 may be indicative of a total of 8 different variables simultaneously.

In some embodiments, the first face of the cube may comprise a first edge indicative of the variable RVSV and a second edge indicative of the variable Ep, and thus the first face is a 2D graph of the relationship between RVSV and Ep, or in other words PPP. The second face of the cube may comprise a first edge indicative of RVSV and a second edge indicative of HR, and therefore be a 2D graph of the relationship between RVSV and HR, or otherwise RVCO. The third face of the cube may comprise a first edge indicative of HR, and a second edge indicative of Ep, and thus be a 2D graph of the relationship between HR and Ep, or otherwise PVR. The first face may have a set of reference lines indicative of PPP. The second face may have a set of reference lines indicative of RVCO. The third face may have a set of reference lines indicative of PVR. Subsequent to two or more data point being plotted in 3D space 530, based on HR, time and/or cardiac cycle, the cube 510 may be indicative of a total of 7 different variables.

In some embodiments, the first face of the cube may comprise a first edge indicative of the variable PPP and a second edge indicative of the variable RVSV, and thus the first face is a 2D graph of the relationship between PPP and RVSV, or in other words RVSW. The second face of the cube may comprise a first edge indicative of RVSV and a second edge indicative of HR, and therefore be a 2D graph of the relationship between RVSV and HR, or otherwise RVCO. The third face of the cube may comprise a first edge indicative of PPP, and a second edge indicative of HR, and thus be a 2D graph of the relationship between PPP and HR, or otherwise RVRPP.

In some embodiments, the first face of the cube may comprise a first edge indicative of the variable PPP and a second edge indicative of the variable RVSVI, and thus the first face is a 2D graph of the relationship between PPP and RVSVI, or in other words RVSWI and/or EpI. The second face of the cube may comprise a first edge indicative of RVSVI and a second edge indicative of HR, and therefore be a 2D graph of the relationship between RVSVI and HR, or otherwise RVCI. The third face of the cube may comprise a first edge indicative of PPP, and a second edge indicative of HR, and thus be a 2D graph of the relationship between PPP and HR, or otherwise RVRPP.

In some embodiments, the first face of the cube may comprise a first edge indicative of the variable PPP and a second edge indicative of the variable RVCO, and thus the first face is a 2D graph of the relationship between PPP and RVCO, or in other words RVP or PVR. The second face of the cube may comprise a first edge indicative of RVCO and a second edge indicative of HR, and therefore be a 2D graph of the relationship between RVCO and HR, or otherwise RVSV. The third face of the cube may comprise a first edge indicative of PPP, and a second edge indicative of HR, and thus be a 2D graph of the relationship between PPP and HR, or otherwise RVRPP.

In some embodiments, the first face of the cube may comprise a first edge indicative of the variable PPP and a second edge indicative of the variable RVCI, and thus the first face is a 2D graph of the relationship between PPP and RVCI, or in other words RVPI or PVRI. The second face of the cube may comprise a first edge indicative of RVCI and a second edge indicative of HR, and therefore be a 2D graph of the relationship between RVCI and HR, or otherwise RVSVI. The third face of the cube may comprise a first edge indicative of PPP, and a second edge indicative of HR, and thus be a 2D graph of the relationship between PPP and HR, or otherwise RVRPP.

In some embodiments, the interactive user interface and the visual representation will be presented based on one or more default presentation style and/or pre-sets. The default presentation style may comprise a set viewing angle, zoom level, colour style and/or palette, rate of plotting (e.g. 1 min in monitoring time=1 second of plotting time) and/or data point grouping (e.g. data point colour(s)).

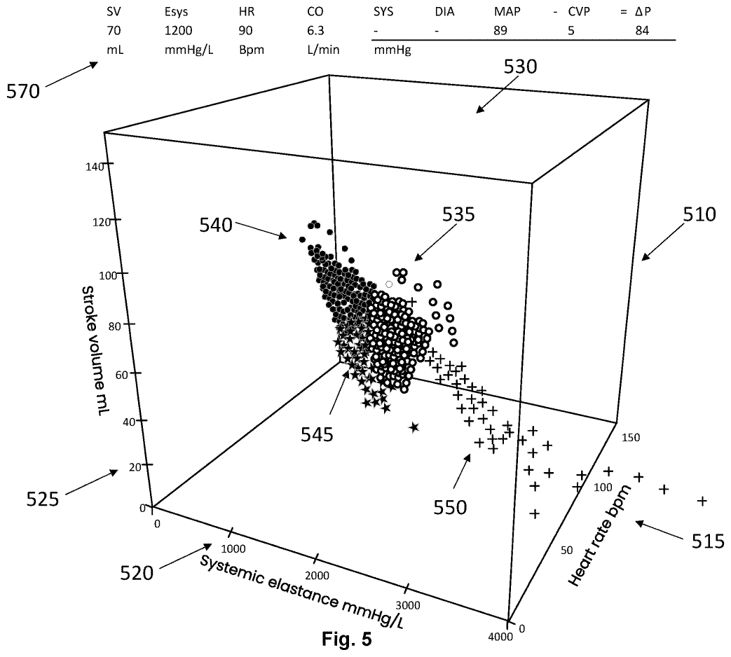

At 435, the data points are plotted in the 3D space (defined 530 in FIG. 5) by the visual representation (cube 510 in FIG. 5) of the interactive user interface. The interactive user interface may also display one or more read outs of the data points. In some embodiments, the data points may be plotted one at a time indicating the time series of the data points. The user interface (not shown) of computing device 235 translates values for the first, second and/or third variables into graphical visual display elements on the display, such as data points on a graph.

In some embodiments, the data points may be plotted evenly overtime, for example one data point per second. When plotted evenly over time, the time variable may be the rate at which a user elects or selects the data points be plotted or the rate at which they were recorded. For example, the user may interact with a plotting rate option element (not shown) of the user interface to select or input a suitable or desired data point plotting rate. The data points may, in some embodiments, be plotted over cardiac cycles or heart rate, such that when a cardiac cycle is shorter or a heart rate is faster, the time between successive data points being plotted in the 3D space is shorter. Similarly, when the cardiac cycle is longer or the heart rate is slower, the time between successive data points is longer, compared to a shorter cardiac cycle or faster heart rate. In some embodiments, the user may alter the rate at which the data points are plotted, for example, by interacting with a plotting rate option element (not shown) of the user interface, regardless of whether the data points are being plotted over time or over cardiac cycle. The rate may be a fixed rate, a variable rate, or an automatically predetermined rate based on one or more inputs or the first, second or third variables and/or health measures. In some embodiments, the frequency of plotting may correlate with a changing colour palette or a sound with frequency varying according to heart rate or CC.

Optionally at 440, in some embodiments where the biometric data is being collected contemporaneously, the steps of 415 to 435 may be performed iteratively to collect and/or determine the first dataset, determine the second dataset, generate the visual representation and display the visual representation as new biometric data is received and/or determined.

The interactive user interface is configured to facilitate orientation of the visual representation (for example, cube) to allow different views of the visual representation and accordingly, the first, second and third health variables and/or measures to be presented. At 440, the system 200 receives an orientation input from the user, such as via the display 260 indicative of a desired change, reorientation, transformation, or alteration of the graphical user interface and/or the visual representation. The orientation input may be, for example, a tap; a swipe; a pinching motion; a click and drag motion such as by a mouse; a scrolling motion, such as performed by a mouse scroll wheel; the selection of a short cut, such as via a physical or onscreen button; and/or any other type of interaction that a user may make with the visual representation, the display 260 and/or the computing device 235.

At 450, the visual representation module 248 receives the orientation input or an indication of the change, reorientation, transformation or alteration of the visual representation from display 260. In some embodiments, the system 200 or display 260 may communicate the orientation input to the visual representation module 248. The computing device 235 may comprise an operating system (not shown), responsible for or otherwise configured to detect user interactions with the user interface and feeding a representation of the detected user interactions to the user interface so that it can respond according to the user interface configuration. Visual representation module 248 may receive the representation of the orientation input and interpret the change, reorientation, transformation or alteration that is to be applied to the interactive user interface and/or visual representation. For example, if the orientation input is a pinching motion on a touch screen, the visual representation module 248 will interpret the pinching motion as an alteration to increase the size of the visual representation for example, zoom into a portion of the visual representation.

In some embodiments, the orientation input may be a selection of a particular face of the visual representation, the first, second or third face, for example. The selection may be, for example, an orientation input that causes the visual representation to rotate towards a particular face of the visual representation, such as by swiping across the screen, clicking and dragging the visual representation with a mouse or by tapping/clicking on a face of the visual representation or one of a plurality of visual representation face selection buttons or shortcuts, for example. The display 260 and/or the visual representation module 248 may be configured to detect and/or track the orientation of the visual representation, and when a particular face of the visual representation reaches a certain position relative to the viewing angle of the user, such as within 20 degrees, within 15 degrees, within 10 degrees, within 8 degrees, within 6 degrees, within 4 degrees or within 2 degrees of the face being substantially perpendicular to the viewing angle of the graphical user interface and interpret this as a selection of a particular face of the visual representation. Or in other words, when the face of the visual representation is substantially parallel to the surface of the display 260, the visual representation module 248 may cause the visual representation to automatically snap to focus on the particular face of the visual representation, causing the particular face of the visual representation to be positioned perpendicular to the viewing angle of the graphical user interface if it was not already so.

Subsequent to receiving the orientation input that causes the visual representation face to snap to focus, the visual representation module 248 is configured to transform the visual representation into the two-dimensional graph representation that is associated with the particular face and variables of that face that the visual representation has been caused to snap to. The visual representation module 248 may further cause one or more axes scales to appear on one or more edges of the 2D graph. In addition, one or more reference lines may be caused to appear on the face of the visual representation. In some embodiments, the visual representation module 248 may plot a subset of the first and second datasets or the third dataset on the two-dimensional graph. The subset may comprise the values associated with the variables of the particular two-dimensional graph.

When the visual representation is transformed into the 2D graph orientation, the user may provide an input to rotate the visual representation away from the particular face of the visual representation that is being focused on, or otherwise the particular two-dimensional graph. Subsequent to receiving the input to rotate the visual representation away from the particular visual representation face, the visual representation module 248 may cause the visual representation to transform from the two-dimensional graph representation into the visual representation. This may include replotting the data points within the interior of the visual representation, removing one or more reference lines from the face of the visual representation and/or adding or removing one or more axes labels/scales.

In some embodiments, the orientation and/or reorientation of the visual representation may not require processing by the visual representation module 248; in this instance, the display 260 may perform the change as indicated by the user input.

At 455, the system 200 may again perform steps 445, 450 and 455 to enable the user to provide additional orientation inputs to change the orientation of the visual representation to provide an intuitive depiction of the health information of the subject 205. In this way, the user, such as a clinical professional, may present, interrogate and/or interpret the information in a variety of ways at will, leading to more comprehensive and/or accurate determinations and/or understandings of a subject's status, thereby leading to better health outcomes for the subject 205.

The described embodiments offer the benefit of presenting health information of a subject 205 in a novel and intuitive way. By simultaneously representing a combination of different variables that are indicative of a subject's 205 health information, in the form of data points plotted within a 3D space, the relationship between the variables, as described by the spatial relationship of the data points, affords the user a clear and readily understandable representation of a large and complex series of data, in a single interactive user interface. Further, the spatial relationship between the data points, and thereby the variables they are representative of, and the time series over which they are plotted, form the basis for additional sources of information, from which the user may derive additional understandings, and therefore determinations about the status, or evolution of the status of the subject 205.

The present disclosures and embodiments combine information, that would previously have been presented either over separate monitoring devices, or over separate graphical representations rendered on the same screen, to simplify the determination of a subject's 205 status, reduce the number of screens and/or graphs necessary to be presented and viewed, freeing up space in potentially space limited environments, such as surgical theatres, enable users to focus their attention on determining the status of a subject 205, and reduce the cognitive load of screen swapping to track a subject's 205 health information.

Further, by presenting the health variables, and the associated health information in a novel way that integrates a spatial element, by the relative positioning of data points and locations of newly plotted data points over time, new understandings of the data, the subject 205 and/or a condition the subject 205 may be experiencing or progress of the condition, may be developed and/or discovered by the users.

In other words, the present disclosures can be said to provide a unifying framework for understanding the variables represented by the data points plotted within the 3D zone, one that may not be readily apparent or even apparent at all if the variables were represented in separate graphical representations. The present disclosures may assist with reconciling data to an overarching clinical paradigm, relating the separate variables, with the values of the variables themselves. For example, the present disclosures may be a useful tool to facilitate medical professionals shifting their clinical understanding of haemodynamics from previous physiological and/or current physiological models, to newer physiological models, as informed by the interactive user interface comprising the visual representation. The present disclosures may achieve this by facilitating the generation of interactive user interfaces to facilitate more comprehensive user understanding of the plotted data.

In some embodiments, the described embodiments allow for ready detection of system defaults or defects. For example, where a plurality of data points are plotted in the 3D space, the system 200 may determine that one or more of the variables of a set of the plurality of data points deviates from an expected zone for the respective variables, and in response, determine that a system defect or default has occurred. For example, the system 200 may determine that stroke volume has fallen below a first threshold, that systemic elastance has exceeded a second threshold, and that heart rate is relatively stable, and may therefore infer or determine that an arterial line has become damped. In response to determining that a system defect has occurred, the system 200 may present a notification on the display screen of the device indicative of the occurrence of the system defect. Values for expected zones and/or thresholds may be stored in memory 239 of the computer device 235, or may be stored and retrieved from database 380, for example.

By way of the present embodiments, the visual representation may be described as simultaneously representing six distinct variables and measures (for example, SV, Esys, HR, SPP, CO, and SVR), indicative of a subject's health information, in addition to representing these across time as a seventh variable. In other words, each data point within the 3D space of the visual representation may represent a unique configuration of six variables and measures, with time as the seventh variable/measure whenever more than one point is plotted. Accordingly, seven variables/measures are represented in a single graphical representation. To present the same number of variables/measures according to prior art methods would require three graphs, which become quite small when on a tablet screen, or require separate screens, and there is necessarily repetition of data across these screens or graphs.

FIG. 5 is an image of a graphical user interface (GUI) 500 as provided by the described embodiments. GUI 500 may be rendered on a smart phone, a tablet computer, laptop, vital sign monitoring computing device, a monitor, a television and/or projector, for example. GUI 500 may comprise visual representation, in this case cube 510, and for example, data readout 570. Data readout 570 may be configured to display the values of one or more of the first variable, second variable and/or third variable, or measures or it may display statistical values derived therefrom. The user interface (not shown) of the computing device 235 may translate the values of the variables of the data points for display via data readout 570.

Cube 510 may comprise one or more variable edges 515, 520, 525, each variable edge 515, 520, 525 being indicative of one of the first variable, second variable or third variable. Cube 510 may also have 3D space 530, constituting the internal volume of cube 510, wherein the one or more data points are plotted. Plotted in 3D space 530 are data points 535. Data points 535, in some embodiments, may be separated into data sub-groups 540, 545, 550.

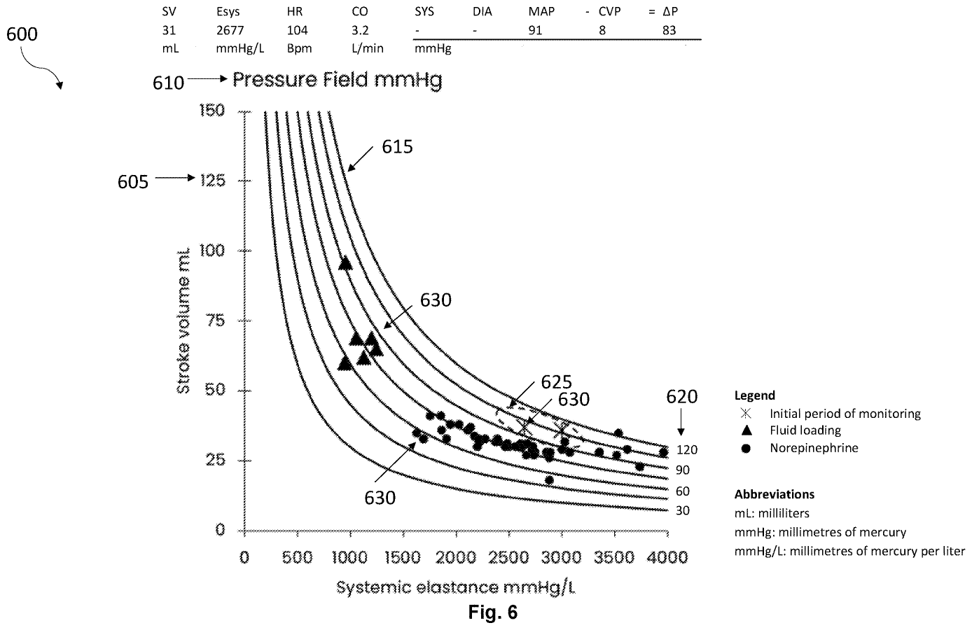

FIG. 6 is a focused view 600 of the GUI 500 of FIG. 5, according to some embodiments. If an orientation input to view a first face of the cube indicative of the variables Esys and SV is received from the user, in some embodiments, the GUI will be caused to snap to 2D graph 605, indicative of SV plotted against Esys. 2D graph 605 may comprise graph title 610, reference lines 615, reference line labels 620, zone 625, data points 630, and/or grid lines (not shown). Reference lines 615 may be configured to aid the user to determine or read/estimate the value(s) of the data points 630 plotted on 2D graph 605.

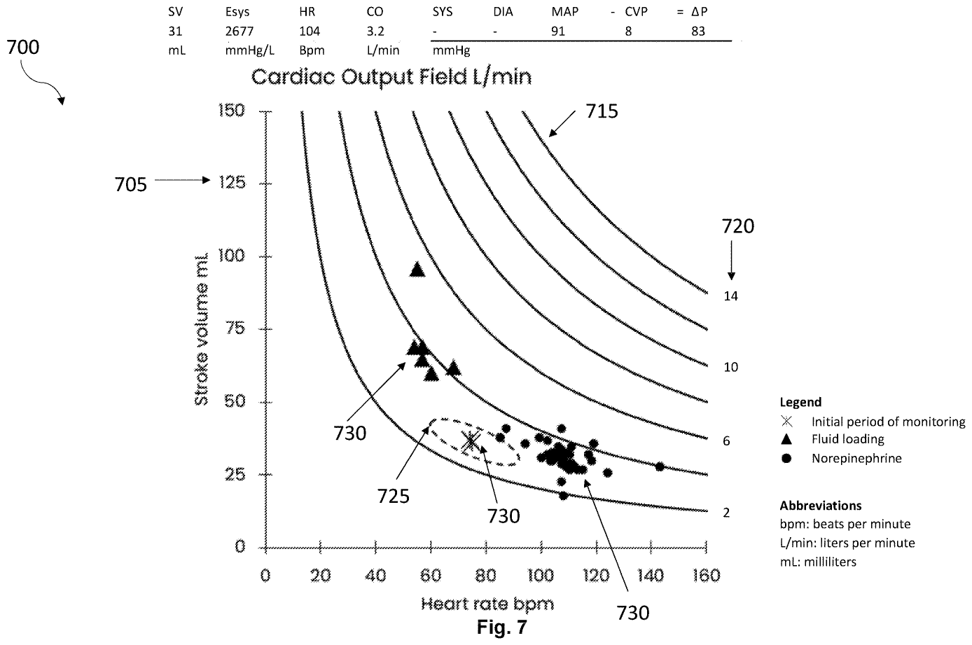

FIG. 7 is a focused view 700 of the GUI 500 of FIG. 5, according to some embodiments. If an orientation input to view a second face of the cube indicative of variables HR and SV is received from the user, in some embodiments, the GUI will be caused to snap to 2D graph 705, indicative of HR against SV. 2D graph 705 may comprise reference lines 715, reference line labels 720, zone 725, data points 730 and/or grid lines (not shown). Reference lines 720 may be configured to aid the user to determine or read/estimate the value(s) of the data points 730 plotted on 2D graph 705.

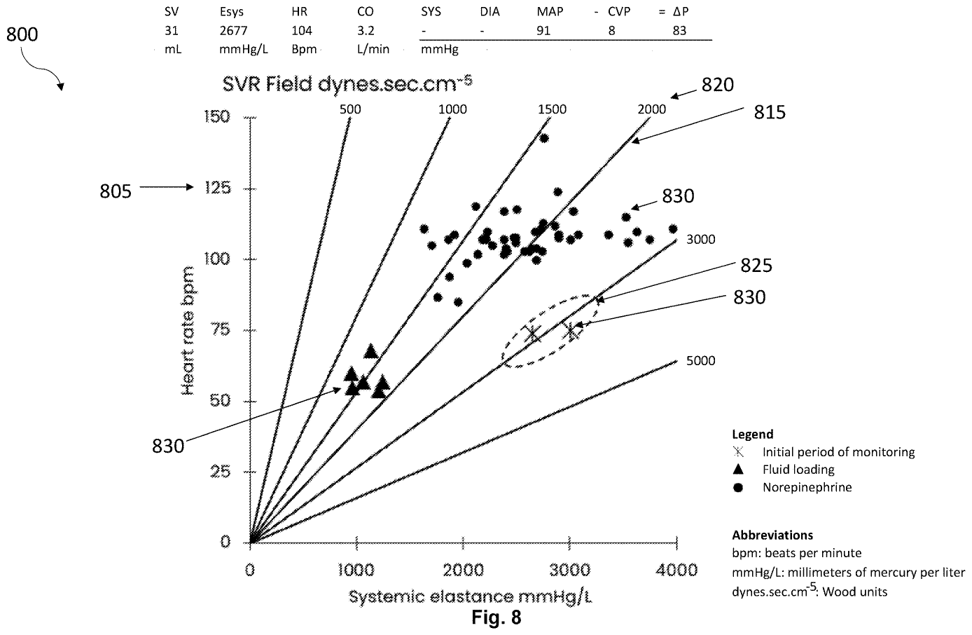

FIG. 8 is a focused view 800 of the GUI 500 of FIG. 5, according to some embodiments. If an orientation input to view a third face of the cube indicative of variables HR and Esys is received from the user, in some embodiments, the GUI will be caused to snap to 2D graph 805, indicative of HR against Esys. 2D graph 805 may comprise reference lines 815, reference line labels 820, zone 825, data points 830, and/or grid lines (not shown). Reference lines 815 may be configured to aid the user to determine or read/estimate the value(s) of the data points 830 plotted on 2D graph 805.

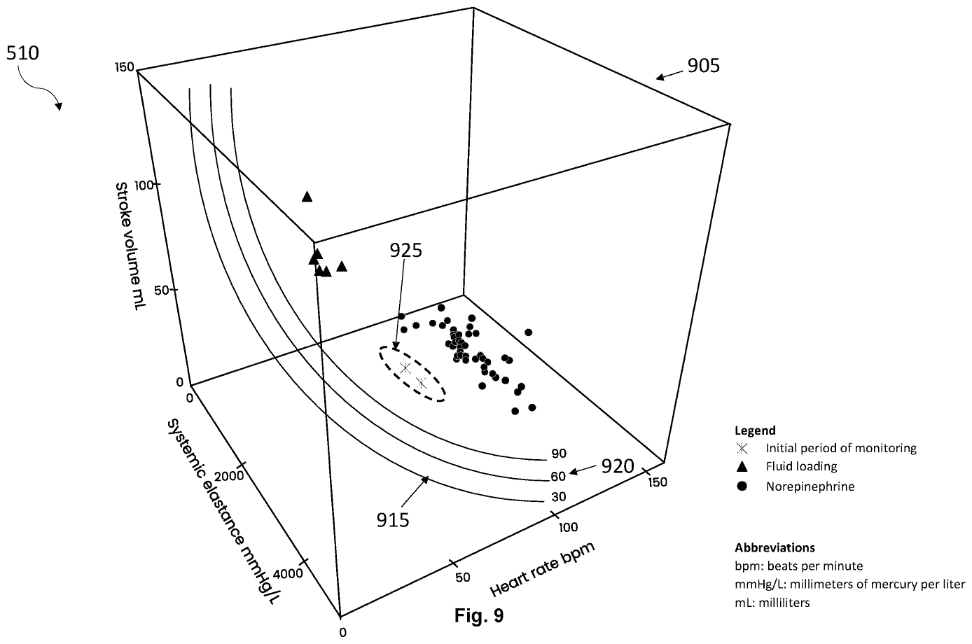

FIG. 9 is an image of cube 510 of FIG. 5, according to some embodiments. In some embodiments, the cube 510 may comprise reference lines 915, reference line labels 920 and zone 925. In FIG. 9, reference lines 715, 815 are not shown. However, in some embodiments, cube 510 may comprise lines 715, 815. In some embodiments, cube 510 comprises any one of, or any combination of two or more of reference lines 615, 715, 815.

Reference lines 615, 715, 815, 915 may or may not be based on the values of data points 630, 730, 830, 535, and may or may not be based on any larger dataset indicative of population level health trends. Zones may be indicative of particular health statuses, such that any data points that are plotted within the zones 625, 725, 825, 925 may be indicative of the particular health status of that zone. For example, zones 625, 725, 825, 925 may be indicative of a healthy ‘normal’ zone for a patient. In some embodiments, zones 625, 725, 825, 925 may be indicative of an acute disease such as tamponade and therefore any data points that fall within zones 625, 725, 825, 925 may be indicative that the subject 205 may be experiencing tamponade. In other words, 2D graphs 605, 705, 805 and/or on a face of cube 910 of FIG. 9 may comprise one or more zones that are demarcated by a particular shape, shaded region, colour, label and/or any other type of indication, which are indicative of a particular health status of the patient, such as normal health, non-normal health and/or a particular condition, such as haemorrhage or tamponade. The one or more zones may be in different locations on 2D graphs 605, 705, 805 and/or on a face of cube 910 of FIG. 9 and/or have different shapes, and/or sizes according to one or more qualities of the subject.

According to some embodiments, 2D graphs 605, 705 and 805 and cube 510 may or may not comprise reference lines 615, 715, 815, 915 and/or zones 625, 725, 825, 925. In some embodiments, reference lines 615, 715, 815, 915 and/or zones 625, 725, 825, 925 may be caused to appear/disappear by an input by the user, such as by interacting with an onscreen user face element, and/or a physical button, switch, or toggle. The grid lines (not shown) may also be rendered in the 3D space 530 of cube 510. For example, grid lines (not shown) may extend throughout the entire volume of 3D space 530 or only a portion of grid lines (not shown) may be rendered proximal to data points 535 plotted in 3D space 530, such that they do not extend to intersect with the edges of cube 510.

Zones 625, 725, 825, 925 may be identified within the cube 510 and the shape of zones 625, 725, 825, 925 may vary according to time. In some embodiments, zones 625, 725, 825, 925 may be safe zones. For example, in some embodiments 625, 725, 825, 925 may be indicative of data points that indicate a safe zone where biometric data of subject 205 may indicate a low possibility of organ injury. This initial safe zone may be very wide for a short period of time, such as five minutes from the time of commencing recordal of the biometric data during contemporaneous recording and presentation of the data, for example. However, the safe zone may gradually shrink so that over a period of time, such as days, hours, minutes and/or seconds the safe zone becomes progressively smaller. Accordingly, the outer boundary of the safe zone, as defined by the data points indicative of the early safe zone, and the inner or otherwise reduced boundary, as defined by data points indicative of later periods of comparatively lower subject safety define the overall extent of a zone, within which biological processes, such as perfusion, can occur. Accordingly, the net risk of organ injury then depends on the cumulative risk of time spent in or out of the zone as defined by the outer boundaries.

In some embodiments, system 200 may determine the positions of the one or more data points 535 in the 3D zone 530 of the cube relative to the safe zone. The system may, in particular, track the positions of the data points 535 relative to the outer boundary of zones 625, 725, 825, 925. The system 200 may track, calculate or otherwise determine an amount of time, based on the heart rate, time and/or cardiac cycles of the subject 205, as indicated by and/or derived from the biometric data, that the first, second and/or third variables have spent outside a safe range of values, as indicated by the zones 625, 725, 825, 925. The system may, responsive to determining the amount of time the first, second and/or third variables have spent outside the safe range of values, determine the risk of injury to the patient.

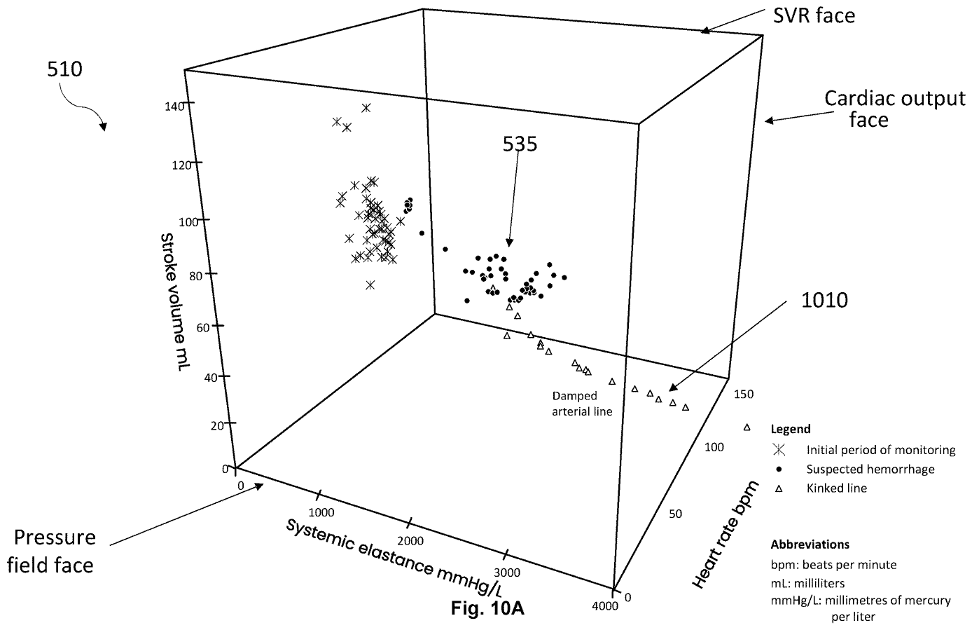

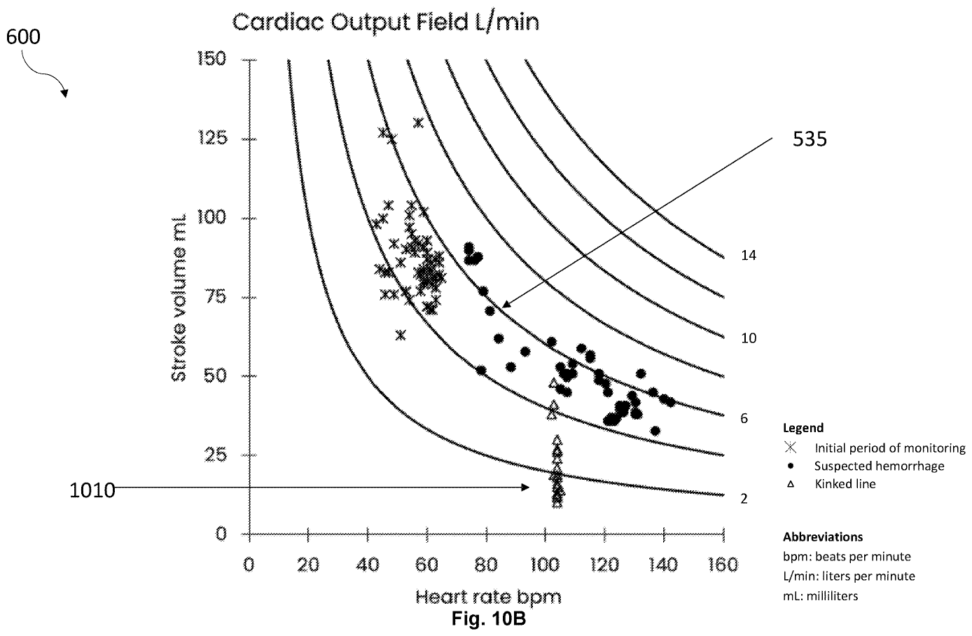

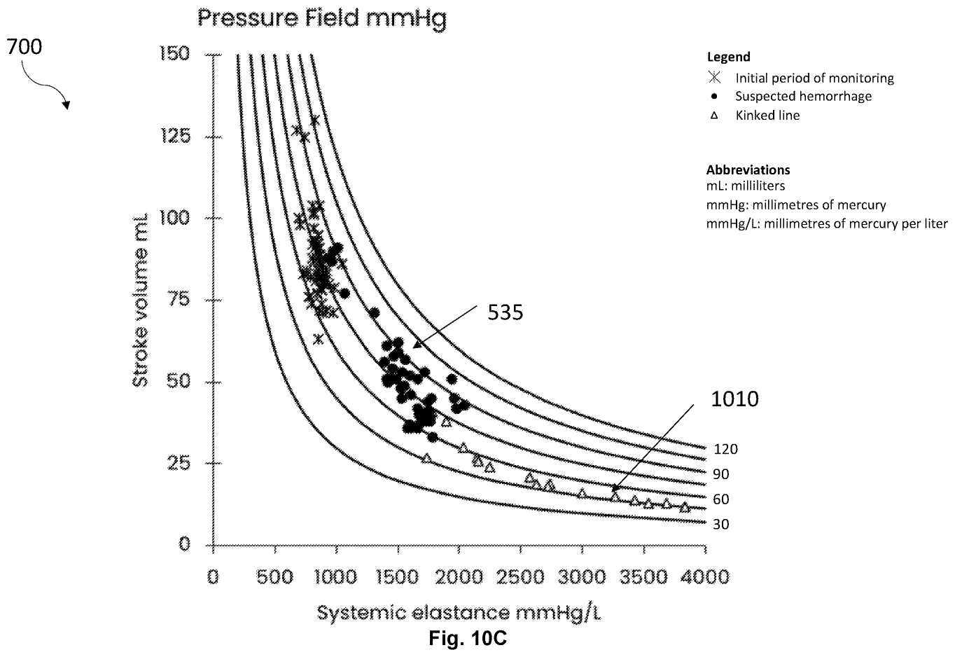

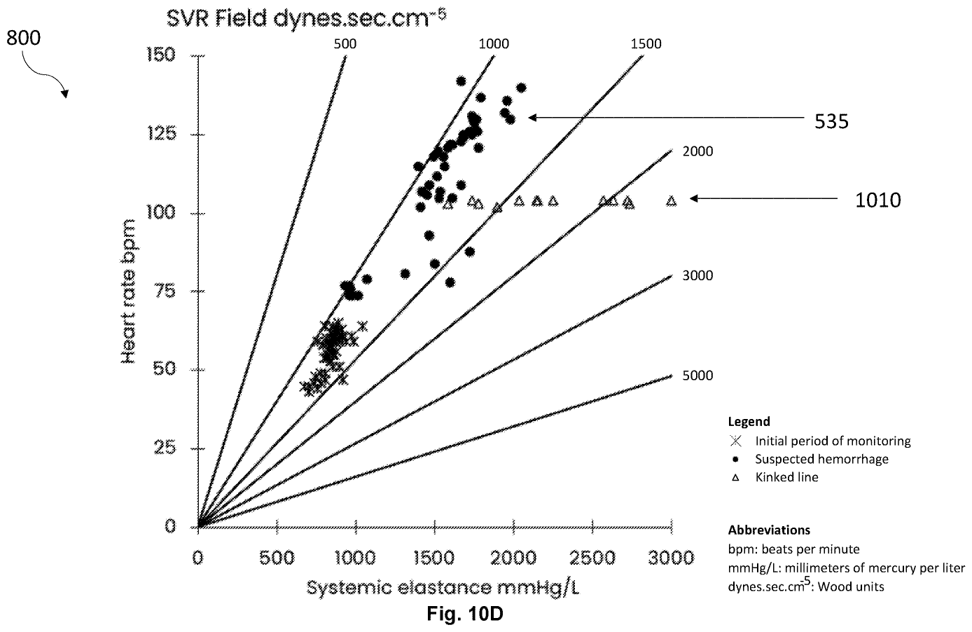

FIG. 10A is image of the cube 510 depicting second data points 1010 indicative of an abnormal system condition, system default and/or system defect, according to some embodiments. The one or more data points 535, in some embodiments, may be considered first data points, and second data points 1010 may not conform to the expected and/or previous trends of the first data points 535. For example, and as depicted in FIG. 10A, while first data points 535 are generally plotted proximal to one another, second data points 1010 extend away from first data points 535 in a substantially uniform line towards the face of the cube 530 defined by SV and HR. The contrast between the location in 3D space 530 of the first and second data points 535, 1010 may mean the second data points 1010 are indicative of an abnormal system condition, system default, and/or system defect, such as may be caused by a damped arterial line (not shown).

FIG. 10B is an image of 2D graph 705, depicting second data points 1010 indicative of an abnormal system condition, system default and/or system defect, according to some embodiments. Similar to that of FIG. 10A, the second data points 1010, as depicted in FIG. 10B, do not conform to the trend and/or positioning of first data points 535. Comparatively, second data points form a substantially vertical line on 2D graph 605. The clear difference between the spatial positioning of first data points 535 and second data points 1010 may be determined to be indicative of an abnormal system condition, system default and/or system defect, either individually, or when taken in combination with the positioning of the second data points 1010 of FIG. 10A.

FIG. 10C is an image of 2D graph 605, depicting abnormal, unusual and/or unexpected data points 1010 indicative of an abnormal system condition, of a system default, and/or a system defect, according to some embodiments. Similar to that of FIG. 10A and FIG. 10B, the second data points do not follow the trend or positioning of first data points 535, and accordingly, may be determined to be indicative of an abnormal system condition, system default and/or system defect, either individually, or when taken in combination with the positioning of one or more of the second data points 1010 of FIG. 10A and/or of FIG. 10B.

FIG. 10D is an image of 2D graph 805, depicting abnormal, unusual and/or unexpected data points 1010 indicative of an abnormal system condition, or a system default, or a system defect, according to some embodiments. The second data points 1010 of FIG. 10D form a substantially horizontal line when plotted on 2D graph 805, and are thus clearly contrasting with the first data points 535, as also plotted on 2D graph 805. Accordingly, second data points 1010 as depicted on FIG. 10D, when either considered alone, or in any combination with one or more of the second data points 1010 of FIGS. 10A to 10C, may be indicative of an abnormal system condition, system default or system defect.

In some embodiments, zones 625, 725, 825, 925 may be indicative of expected values of the first, second and/or third variables and/or measures. For example, expected values may be values that are within a predetermined range of values that are indicative of one or more health statuses of a patient or subject. In some embodiments, system 200 may be configured to determine that one or more variables of the data points 535 deviates from the expected zones 625, 725, 825, 925. Responsive to determining that one or more of the variables of the data points 535 deviates from the expected zone, determining that a system defect has occurred. A system defect may be a malfunction of one or more devices, and/or one or more devices may be impeded in some way. For example, an arterial line may be kinked, blocked, twisted or otherwise damped or arranged in a way which restricts and/or stops it functioning in its intended fashion.

Determining that one or more of the variables has deviated from the expected zones 625, 725, 825, 925 may comprise determining that stroke volume has fallen below a first threshold, that systemic elastance has exceeded a second threshold, and that heart rate is relatively stable; and

The disclosed embodiments provide for a conceptual way of understanding, for example, perfusion, as represented by the plotted data points when the interactive user interface is displaying the cube, with the detail needed to guide user decision-making, such as the reference lines that are super imposed when the cube is transformed into the 2D graph representation, for example. That is, the 3D representation provides a holistic view of the circulation and its response to specific disease states and interventions. It therefore provides greater diagnostic clarity than any single side of the 3D representation, while also enabling the user to drill down or focus on the particular aspect of the circulation most affected by disease or otherwise in need of close management.

The following case studies provide context for the applicability and usefulness of the described embodiments with reference to the data depicted in FIGS. 6 to 10.

### Case Study 1

A patient underwent major surgery. The patient's blood pressure, CO, HR, SV, and SVR were monitored during the period of surgery.

Following the start of general anaesthesia, the patient was fluid loaded with the goal of maintaining blood pressure. This quickly resulted in instability in blood pressure, so that fluid loading was stopped and a norepinephrine infusion commenced. This improved stability in blood pressure (albeit with a high heart rate) and this management approach was maintained for the remaining period of surgery. Following surgery, the patient had an extended stay in ICU with multiple complications related to organ dysfunction and died.

Following the case, the monitoring data was visualised in the cube visualisation (see FIGS. 6 to 9). The patient's pre-induction zone is displayed by the hashed symbols 630.

Following the start of general anaesthesia, the fluid loading resulted in significant displacement from the patient's pre-induction zone with increased SV and a decreased E_{sys} indicating vasodilation (see the triangle dots at 630). The norepinephrine infusion resulted in displacement in a different direction: SV returned to roughly near the pre-induction zone, but E_{sys} was significantly higher and lower at times, and heart rate became very elevated (see the round dots at 630).

The visual representation or cube is expected to have assisted in titration of fluids and drugs to better approximate the patient's pre-induction haemodynamic patterns. It is hypothesised that this would have resulted in an improved patient outcome.

### Case Study 2

A patient underwent major surgery and afterwards was admitted to the ICU. The patient's blood pressure, CO, HR, SV, and SVR were monitored during the period of surgery and in the ICU.

The patient suffered post-operative haemorrhage. There was a delay in definitively treating the haemorrhage via reoperation because the patient's haemodynamics appeared to be relatively well maintained (with fluids and vasoactive drugs) despite the haemorrhage. That is, blood pressure, CO, and SVR were relatively well maintained albeit with a high heart rate. Reoperation was eventually undertaken but during the period of haemorrhage there had been significant damage to the vasculature (as a result of vasoconstriction) and as a consequence of this damage the patient experienced further complications and died.

Following the case, the monitoring data was visualised in the cube visualisation (see FIGS. 10A, 10B, 10C, and 10D).

The initial period of care in ICU is represented by the hashed symbols in FIGS. 10A, 10B, 10C, and 10D.

For the period of haemorrhage, the cube visualisation demonstrates the significantly abnormal pattern of haemodynamic function associated with the period of haemorrhage (see the round dots in FIGS. 10A, 10B, 10C, and 10D). FIG. 10A and FIG. 10C show that SV is decreased and the increased E_{sys} values indicate that blood pressure was maintained by severe vasoconstriction. FIG. 10A and FIG. 10B demonstrate that CO was maintained by a tachycardia. FIG. 10A and FIG. 10D demonstrate that the ‘normal’ values in SVR are the result of the increased E_{sys} values being offset by the increased HR.

In summary, there was significant abnormal displacement from the patient's normal zone of haemodynamic function with the visual representation or cube visualisation indicating an underfilled heart coupled with marked vasoconstriction. The visual representation or cube visualisation would have assisted with early diagnosis of the haemorrhage and would have supported the argument for earlier return to the operating room. It is hypothesised that this would have resulted in an improved patient outcome.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method comprising:
determining one or more data points of a time series, each of the one or more data points comprising values for each of a first, second and third variable, each of the first, second and third variables being indicative of health information of a subject;
providing an interactive user interface on a display screen of a device, the interactive user interface comprising a visual representation defining a 3D space, wherein a first edge of the visual representation is representative of the first variable, a second edge of the visual representation is representative of the second variable, and a third edge of the visual representation is representative of the third variable; and
plotting the one or more data points in the 3D space such that a first face of the visual representation defined by the first and second edges depicts a first health measure defined by a function of the first and second variables, a second face of the visual representation defined by the first and third edges depicts a second health measure defined by a function of the first and third variables, and a third face of the visual representation defined by the second and third edges depicts a third health measure defined by a function of the first and third variables,
wherein the interactive user interface is configured to facilitate orientation of the visual representation to allow different views of the visual representation and accordingly, the first, second and third health variables and/or measures to be presented,
receiving an orientation input from a user via the interactive user interface; and
responsive to receiving the orientation input from a user via the interactive user interface, orientating the visual representation in accordance with the orientation input.

2. The method of claim 1, wherein plotting the one or more data points comprises plotting the one or more data points in time sequence order.

3. The method of claim 1, wherein the one or more data points are measured at successive equally spaced points in time.

4. The method of claim 1, wherein the one or more data points are plotted based on heart rate and/or cardiac cycles.

5. The method of claim 1, wherein collectively, the first, second and third variables and/or health measures depicted by the visual representation represent perfusion or haemodynamic function in the body of the subject.

6. The method of claim 1, wherein:
the first variable is indicative of stroke volume (SV), the second variable is indicative of heart rate (HR), and the first health measure is indicative of cardiac output (CO); or
the first variable is indicative of stroke volume (SV), the third variable is indicative of systemic elastance (Esys), and the second health measure is indicative of systemic perfusion pressure (SPP); or
the second variable is indicative heart rate (HR), the third variable is indicative of systemic elastance (Esys), and the third health measure is systemic vascular resistance (SVR); or
the first variable is indicative of stroke volume (SV), the third variable is indicative of effective arterial elastance (Ea), and the second health measure is indicative of mean arterial pressure (MAP); or
the second variable is indicative of heart rate (HR), the third variable is indicative of effective arterial elastance (Ea), and the third health measure is total peripheral resistance (TPR); or
the first variable is indicative of systemic perfusion pressure (SPP), the second variable is indicative of indexed cardiac output (CI) and the first health measure is indicative of indexed cardiac power (CPI); or
the first variable is indicative of systemic perfusion pressure (SPP), the third variable is indicative of heart rate (HR) and the second health measure is indicative of rate pressure product (RPP); or
the second variable is indicative of indexed cardiac output (CI), the third variable is indicative of heart rate (HR) and the third health measure is indicative of indexed stroke volume (SVI) or;
the first variable is indicative of systemic perfusion pressure (SPP), the second variable is indicative of cardiac output (CO) and the first health measure is indicative of cardiac power (CP); or
the second variable is indicative of cardiac output (CO), the third variable is indicative of heart rate (HR), and the second health measure is indicative of stroke volume (SV); or
the first variable is indicative of systemic perfusion pressure (SPP), the third variable is indicative of heart rate (HR), and the third health measure is indicative of rate pressure product (RPP); or
the first variable is indicative of systemic perfusion pressure (SPP), the second variable is indicative of indexed stroke volume (SVI), and the first health measure is indicative of indexed left ventricular stroke work (LVSWI); or
the second variable is indicative of indexed stroke volume (SVI), the third variable is indicative of heart rate (HR), and the second health measure is indicative of indexed cardiac output (CI); or
the first variable is indicative of systemic perfusion pressure (SPP), the second variable is indicative of stroke volume (SV), and the first health measure is indicative of left ventricular stroke work (LVSW); or
the second variable is indicative of stroke volume (SV), the third variable is indicative of heart rate (HR), and the second health measure is indicative of cardiac output (CO); or
the first variable is indicative of right ventricle stroke volume (RVSV) as the first variable, the second variable is indicative of heart rate (HR), and the first health measure is indicative of right ventricle cardiac output (RVCO); or
the first variable is indicative of right ventricle stroke volume (RVSV), the third variable is indicative of pulmonary elastance (Ep) and the second health measure is indicative of pulmonary perfusion pressure (PPP); or
the second variable is indicative of heart rate (HR), the third variable is indicative of pulmonary elastance (Ep) and the third health measure is indicative of pulmonary vascular resistance (PVR); or
the first variable is indicative of pulmonary perfusion pressure (PPP), the second variable is indicative of right ventricle stroke volume (RVSV), and the first health measure is indicative of right ventricle stroke work (RVSW) or pulmonary elastance (Ep); or
the first variable is pulmonary perfusion pressure (PPP), the third variable is indicative of heart rate (HR) and the second health measure is indicative of right ventricle rate pressure product (RVRPP); or
the second variable is indicative of right ventricle stroke volume (RVSV), the third variable is indicative of heart rate (HR) and the third health measure is indicative of right ventricle cardiac output (RVCO); or
the first variable is indicative of pulmonary perfusion pressure (PPP), the second variable is indicative of right ventricle stroke volume index (RVSVI), and the first health measure is indicative of right ventricle stroke work index (RVSWI); or
the second variable is indicative of right ventricle stroke volume index (RVSVI), the third variable is indicative of heart rate (HR) and the third health measure is indicative of right ventricle cardiac output index (RVCI); or
the first variable is indicative of pulmonary perfusion pressure (PPP) as the first variable, the second variable is indicative of right ventricle cardiac output (RVCO), and the first health measure is indicative of pulmonary vascular resistance (PVR) or right ventricle power (RVP); or
the second variable is indicative of right ventricle cardiac output (RVCO), the third variable is indicative of heart rate (HR) and the third health measure is indicative of the right ventricle stroke volume (RVSV); or
the first variable is indicative of pulmonary perfusion pressure (PPP), the second variable is indicative of right ventricle cardiac index (RVCI), and the first health measure is indicative of pulmonary vascular resistance index (PVRI) or right ventricle power index (RVPI); or
the second variable is indicative of right ventricle cardiac index (RVCI), the third variable is indicative of heart rate (HR) and the third health measure is indicative of the right ventricle stroke volume index (RVSVI).

7. The method of claim 1, wherein the orientation input is a selection of one of the first, second or third face of the visual representation:
responsive to receiving the selection, causing the visual representation to depict, on one of the first, second or third face of the visual representation, a two-dimensional graph;
plotting on the two-dimensional graph the data points, wherein the data points are indicative of the variables associated with the first, second or third face of the visual representation; and
wherein the two-dimensional graph is indicative of one of the first, second or third health measures associated with the first, second or third face of the visual representation respectively.

8. The method of claim 7, comprising plotting at least a first reference line on the two-dimensional graph, the at least a first reference line configured to: (i) aid the user to determine value(s) of the one or more data points plotted on the two-dimensional graph; or (ii) represent one or more health measures relative to the one or more data points plotted on the two-dimensional graph.

9. The method of claim 1, comprising plotting at least a first reference line on each of the first, second and/or third face of the visual representation, wherein the at least first reference lines of the first, second and/or third faces are indicative of different health measures, and wherein the at least first reference line is configured to (i) aid the user to determine value(s) of the one or more data points plotted on the two-dimensional graph; or (ii) represent one or more health measures relative to the one or more data points plotted on the respective first, second and/or third face.

10. The method of claim 8, wherein when the at least first reference line is configured to represent one or more health measures, the one or more health measures comprising one or more of:
systemic perfusion pressure (SPP);
cardiac output (CO);
systemic vascular resistance (SVR);
mean arterial pressure (MAP);
total peripheral resistance (TPR);
cardiac index (CI);
systemic vascular resistance index (SVRI);
total peripheral resistance index (TPRI);
pulmonary perfusion pressure (PPP);
right ventricle cardiac output (RVCO);
pulmonary vascular resistance (PVR);
pulmonary vascular resistance index (PVRI);
right ventricle cardiac index (RVCI);
cardiac power (CP);
cardiac power index (CPI);
rate pressure product (RPP);
left ventricle stroke work (LVSW);
left ventricle stroke work index (LVSWI);
right ventricle stroke work (RVSW);
right ventricle stroke work index (RVSWI);
right ventricle power (RVP);
right ventricle power index (RVPI); or
right ventricle rate pressure product (RVRPP).

11. The method of claim 1, comprising plotting a second reference line on each of the first, second and/or third face of the visual representation, wherein the second reference lines of the first, second and/or third faces are indicative of different health measures, and wherein the second reference line is configured to (i) aid the user to determine value(s) of the one or more data points plotted on the two-dimensional graph; or (ii) represent one or more health measures relative to the one or more data points plotted on the respective first, second and/or third face.

12. (canceled)

13. The method of claim 1, wherein the one or more data points are determined from biometric data that is indicative of one or more of variables or measures:
systemic vascular resistance (SVR);
heart rate (HR);
central venous pressure (CVP);
mean arterial pressure (MAP);
stroke volume (SV);
systemic perfusion pressure (SPP);
systemic elastance (Esys);
cardiac output (CO);
total peripheral resistance (TPR);
effective arterial elastance (Ea);
cardiac cycle time (CC);
mean pulmonary arterial pressure (MPAP);
pulmonary capillary wedge pressure (PCWP);
left atrial pressure (LAP);
pulmonary perfusion pressure (PPP);
pulmonary vascular resistance (PVR);
pulmonary elastance (Ep);
right ventricle stroke volume (RVSV);
right ventricle cardiac output (RVCO);
indexed stroke volume (SVI);
indexed systemic vascular resistance (SVRI);
indexed cardiac output (CI);
indexed systemic elastance (EsysI);
indexed effective arterial elastance (EaI);
indexed total peripheral resistance (TPRI);
indexed right ventricle stroke volume (RVSVI);
indexed right ventricle cardiac output (RVCI);
indexed pulmonary vascular resistance (PVRI); and
indexed pulmonary elastance (EpI);
wherein the one or more indexed variables is indexed based on a body surface area (BSA) of the subject.

14. (canceled)

15. The method of claim 1, wherein the 3D space further comprises a safe zone, being indicative of a range of safe values of the first, second and/or third variables, the safe zone comprising an outer boundary and being indicative of a region of the 3D space associated with a low possibility of injury to the subject.

16. The method of claim 15, wherein the method further comprises:
determining the positions of the one or more data points in the 3D space relative to the safe zone;
determining, based on the positions of the one or more data points proximal to and/or outside of the outer boundary of the safe zone, an amount of time the first, second and/or third variables have been outside the range of safe values;
wherein the amount of time is indicative of a risk of injury to the subject.

17. The method of claim 16, the method further comprising, determining, based on the amount of time the first, second and/or third variables have been outside the range of safe values a level of risk that the subject will be injured.

18. The method according to claim 1, wherein plotting the one or more data points in the 3D space comprises plotting at least a plurality of data points and wherein the method further comprises:
determining that one or more of the variables of a set of the plurality of data points deviate from an expected zone for the respective variables; and
responsive to determining that the one or more of the variables of the set of the plurality of data points deviate from the expected zone, determining that a system defect has occurred.

19. The method of claim 18, wherein determining that one or more of the variables of a set of the plurality of data points deviate from an expected zone for the respective variables comprises:
determining that stroke volume has fallen below a first threshold, that systemic elastance has exceeded a second threshold, and that heart rate is relatively stable; and
responsive to determining that stroke volume has fallen below a first threshold, that systemic elastance has exceeded a second threshold, and that heart rate is relatively stable, determining that an arterial line has become damped.

20. The method of claim 18 wherein responsive to determining that a system defect has occurred, presenting a notification on the display screen of the device indicative of the occurrence of the system defect.

21. (canceled)

22. A system for providing an interactive user interface, the system comprising:
a computing device configured to determine one or more data points of a time series, each of the one or more data points comprising values for each of a first, second and third variable, each of the first, second and third variables being indicative of health information of a subject;
a display screen of a device, configured to display the interactive user interface, the interactive user interface comprising:
a visual representation defining a 3D space, wherein a first edge of the visual representation is representative of the first variable, a second edge of the visual representation is representative of the second variable, and a third edge of the visual representation is representative of the third variable, wherein the one or more data points are plotted in the 3D space such that a first face of the visual representation defined by the first and second edges depicts a first health measure defined by a function of the first and second variables, a second face of the visual representation defined by the first and third edges depicts a second health measure defined by a function of the first and third variables, and a third face of the visual representation defined by the second and third edges depicts a third health measure defined by a function of the first and third variables; and
wherein the interactive user interface is configured to:
facilitate orientation of the visual representation to allow different views of the visual representation and accordingly, the first, second and third health measures to be presented;
receive an orientation input from a user via the interactive user interface; and
in response to receiving the orientation input from the user via the device, orient the visual representation in accordance with the orientation input.

23. (canceled)

24. A non-transient computer-readable storage medium comprising executable code, which when executed by one or more processors, are configured to perform operations including:
determining one or more data points of a time series, each of the one or more data points comprising values for each of a first, second and third variable, each of the first, second and third variables being indicative of health information of a subject;
providing an interactive user interface on a display screen of a device, the interactive user interface comprising a visual representation defining a 3D space, wherein a first edge of the visual representation is representative of the first variable, a second edge of the visual representation is representative of the second variable, and a third edge of the visual representation is representative of the third variable; and
plotting the one or more data points in the 3D space such that a first face of the visual representation defined by the first and second edges depicts a first health measure defined by a function of the first and second variables, a second face of the visual representation defined by the first and third edges depicts a second health measure defined by a function of the first and third variables, and a third face of the visual representation defined by the second and third edges depicts a third health measure defined by a function of the first and third variables;
wherein the interactive user interface is configured to facilitate orientation of the visual representation to allow different views of the visual representation and accordingly, the first, second and third health variables and/or measures to be presented;
receiving an orientation input from a user via the interactive user interface; and
responsive to receiving the orientation input from the user via the interactive user interface, orientating the visual representation in accordance with the orientation input.

25. (canceled)
